(19) United States
(12) Patent Application Publication
(10) Pub. No.: US 2025/0041377 A1
(43) Pub. Date: Feb. 6, 2025

(54) PHARMACEUTICAL USE

(71) Applicant: ENLITISA (SHANGHAI) PHARMACEUTICAL CO., LTD., SHANGHAI (CN)

(72) Inventors: Ming GU, Jiangyin (CN); Bengt Ingemar SAMUELSSON, Stockholm (SE); Jan-Christer JANSON, Uppsala (SE)

(21) Appl. No.: 18/612,989

(22) Filed: Mar. 21, 2024

Related U.S. Application Data
(63) Continuation of application No. 17/058,482, filed on Nov. 24, 2020, now abandoned.

(30) Foreign Application Priority Data
May 28, 2018 (WO) PCT/CN2018/088701

(51) Int. Cl. A61K 38/08; A61K 9/00; A61K 31/47; A61P 11/00; A61P 11/04; A61P 17/00; A61P 17/02; A61P 37/06; C07K 7/06

(52) U.S. Cl. CPC A61K 38/08; A61K 9/0014; A61K 9/0073; A61K 31/47; A61P 11/00; A61P 11/04; A61P 17/00; A61P 17/02; A61P 37/06; C07K 7/06

(57) ABSTRACT

There is provided a peptide of the sequence Ala-Lys-Pro-Ser-Tyr-Hyp-Hyp-Thr-Tyr-Lys, or a salt thereof, for use as a pharmaceutical. The peptide is particularly useful in the treatment of conditions characterised by inflammation, including wounds, burns, hemorrhoids, psoriasis, acne, 5 atopic dermatitis, COPD ulcerative colitis and IPF.

This application is a continuation of U.S. application Ser. No. 17/058,482, which is a national stage application under 35 U.S.C. § 371 of PCT Application No. PCT/CN2019/088593, filed May 27, 2019, which is a continuation-in-part of (and claims priority benefit to) PCT Application No. PCT/CN2018/088701, filed May 28, 2018.

### SEQUENCE LISTING

This application contains a computer readable Sequence Listing which has been submitted electronically in XML format and is hereby incorporated by reference in its entirety. Said XML copy, created on Mar. 21, 2024, is named 147432001260.xml and is 4,338 bytes in size.

### FIELD OF THE INVENTION

This invention relates to the new use of a known compound in human medicine, and to pharmaceutical compositions comprising it. In particular, the invention relates to the use of the compound and those compositions in the treatment of inflammation.

### BACKGROUND AND PRIOR ART

Inflammation is typically characterised as a localised tissue response to e.g. invasion of microorganisms, certain antigens, damaged cells or physical and/or chemical factors. The inflammatory response is normally a protective mechanism which serves to destroy, dilute or sequester both the injurious agent and the injured tissue, as well as to initiate tissue healing.

Inflammation may result from physical trauma, infection, some chronic diseases (e.g. psoriasis and autoimmune diseases, such as rheumatoid arthritis) and/or chemical and/or physiological reactions to external stimuli (e.g. as part of an allergic response). A complex series of events may be involved, in which inflammatory mediators increase blood flow and dilation of local blood vessels, resulting in redness and heat, the exudation of fluids, often resulting in localised swelling, leukocytic migration into the inflamed area, and pain.

Many conditions/disorders are characterized by, and/or are caused by, abnormal, tissue-damaging inflammation. Such conditions are typically characterized by activation of immune defence mechanisms, resulting in an effect that is more harmful than beneficial to the host, and are generally associated with varying degrees of tissue redness or hyperemia, swelling, hyperthermia, pain, itching, cell death, tissue destruction, cell proliferation and/or loss of function. Examples include inflammatory bowel diseases, rheumatoid arthritis, multiple sclerosis, psoriasis, glomerulonephritis and transplant rejection.

Typically, a complex series of events results in inflammatory changes such as increased blood flow through dilation of local blood vessels, resulting in redness and heat, the extravasation of leukocytes and plasma, often resulting in localised swelling, activation of sensory nerves (resulting in pain in some tissues) and loss of function. These inflammatory changes are triggered by a cascade of cellular and biochemical events involving cells like neutrophils, monocytes, macrophages and lymphocytes together with inflammatory mediators such as vasoactive amines, cytokines, complement factors and reactive oxygen species.

Amongst other things, inflammation plays a key role in the wound healing process. Wounds and burns can therefore be classified as conditions with which inflammation is associated. Traditional thinking in the art is that antiinflammatory drugs should not be applied directly to open wounds, as this would be detrimental to the progress of wound healing.

Mussel adhesive protein (MAP), also known as Mytilus edulis foot protein (mefp), is a protein secreted by marine shellfish species, such as Mytilus edulis, Mytilus coruscus and Perna viridis. The adhesive protein is secreted by mussels from the byssus gland where it is produced and stored. When secreted on a surface of a solid, such as a rock, but also other solid objects, such as metals, wood, glass, etc., a water-proof bond is formed which fixes the mussel to the solid object. Mussels are typically attached, in groups, to coastal reefs or to the bottoms of ships. The bond is incredibly strong, having the ability to resist wave impacts in coastal waters.

Studies on Mytilus edulis, Mytilus galloprovincialis, Mytilus californias and Perna viridis have thus far identified eleven separate adhesive protein subtypes derived from mussels: mfp-1 (sometimes referred to as “mefp-1”, hereinafter used interchangeably), mfp-2/mefp-2, mfp-3/mefp-3, mfp-4/mefp-4, mfp-5/mefp-5, mfp-6/mefp-6; the collagens pre-COL-P, pre-COL-D and pre-COL-NG; and the mussel feet matrix proteins PTMP (proximal thread matrix protein) and DTMP (distal proximal thread matrix protein). See, for example, Zhu et al, Advances in Marine Science, 32, 560 (2014) and Gao et al, Journal of Anhui Agr. Sci., 39, 19860 (2011)).

All mussel adhesive proteins, including sub-types thereof, have two structural characteristics, in that they comprise: (1) lysine, such that the protein carries a high positive charge loading (due to the NH₂ termini); (2) 3,4-dihydroxyphenylalanine (DOPA, dopamine), the catechol part of which is responsible for the formation of strong covalent bonds and consequently the ability of mussel adhesive proteins to bind to solid surfaces.

Products based on mussel adhesive protein products are presently used in a limited number of fields (including micro-cellular bonding, as tissue bonding agents and the treatment of wounds and burns). Commercial products are either directly used as a solution of mussel adhesive protein or are stored as a freeze-dried powder for dissolution prior to use.

A significant portion of mefp-1 consists of 70 to 90 tandem repeats of the decapeptide: Ala-Lys-Pro-Ser-Tyr-Hyp-Hyp-Thr-DOPA-Lys (SEQ ID NO: 1) (see Waite, Int. J. Adhesion and Adhesives, 7, 9 (1987)). This decapeptide sequence may by isolated as a low molecular weight derivative of naturally-occurring MAPs, or may be synthesized, for example as described by Yamamoto in J. Chem. Soc., Perkin Trans. 1, 613 (1987). See also Dalsin et al, J. Am. Chem. Soc., 125, 4253 (2003).

The DOPA residue is considered to be essential to the activity of MAPs, such as mefp-1. There is no suggestion in the art that its complete replacement with a different amino acid, such as tyrosine, might result in a retention of MAP's properties, in a physico-chemical, or in a biological, sense.

There is certainly no suggestion in the art that an isolated decapeptide containing no DOPA residues, which has been disclosed only as a model compound by, for example, Kanyalkar et al in Biomaterials, 389 (2002) and Belli et al in Dental Materials, 26, e125 (2010), would or even could possess the same, or even similar, properties to MAP. Surprisingly, we have found that an isolated decapeptide with a different structure to the repeat decapeptide unit in mefp-1 is useful in the treatment of inflammation. See also U.S. Pat. No. 5,616,311 and WO 96/39128.

### DISCLOSURE OF THE INVENTION

According to the invention, there is provided an (isolated) peptide compound comprising, or preferably consisting of, the sequence
Ala-Lys-Pro-Ser-Tyr-Hyp-Hyp-Thr-Tyr-Lys, (SEQ ID NO: 2) where each Hyp is 3-hydroxyproline or 4-hydroxyproline,
   a regioisomer, a stereoisomer, or a salt, thereof, for use in medicine and/or as a pharmaceutical, for example in the treatment of inflammation an inflammatory disorder, and/or of a disorder characterised by inflammation.

There is further provided an (isolated) peptide compound of the above-mentioned sequence for use in veterinary science and/or as a cosmetic. The compounds (including regioisomers and stereoisomer) and salts thereof that are disclosed herein for the uses mentioned herein are referred to hereinafter together as “the compounds of the invention”.

Compounds of the invention, whether in the form of salts or otherwise, include regioisomers within certain amino acids in the above structure (e.g. Hyp and Tyr), as well as mixtures of such regioisomers.

For example, included within the definition of Tyr are, not only tyrosine (4-hydroxyphenylalanine), but also 2- and 3-hydroxyphenylalanine, and included within the definition of Hyp are 4-hydroxyproline, 3-hydroxyproline and 5-hydroxyproline. The Hyp residue that is adjacent to the Tyr residue in a compound of the invention may be 3-hydroxyproline, and the Hyp residue that is adjacent to the Thr residue in a compound of the invention may be 4-hydroxyproline. It is more preferred that the Hyp residues are both 4-hydroxyproline.

Thus, compounds of the invention not in the form of salt may have the following specific chemical structure according to SEQ ID NO: 2, where each Hyp is 4-hydroxyproline as shown below:

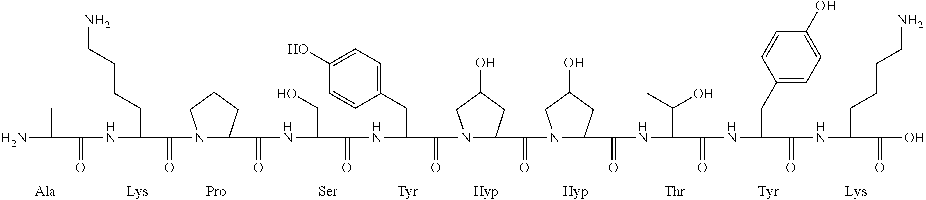 [SMILES: CC(N)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)cc1)C(=O)NC(CCCCN)C(=O)O)C(C)O]

Also, in addition to the standard central carbon atom of the amino acids in the above sequence (which is without exception in the L-configuration), certain amino acids in the sequence comprise further chiral carbon atoms. All such stereoisomers and mixtures (including racemic mixtures) thereof are included within the scope of the invention. In respect, included within the definition of Hyp are trans-4-hydroxy-L-proline, cis-4-hydroxy-L-proline, trans-3-hydroxy-L-proline, cis-3-hydroxy-L-proline trans-5-hydroxy-L-proline and cis-5-hydroxy-L-proline, however we prefer that the Hyp that is employed in compounds of the invention is 4-hydroxy-L-proline.

Compounds of the invention may be in the form of salts. Salts that may be mentioned include pharmaceutically-acceptable and/or cosmetically-acceptable salts, such as pharmaceutically- and/or cosmetically-acceptable acid addition salts and base addition salts. Such salts may be formed by conventional means, for example by reaction of free peptide with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of active ingredient in the form of a salt with another counter-ion, for example using a suitable ion exchange resin.

Preferred salts include, for example, hydrochloride, bisulfate, maleate, mesylate, tosylate, alkaline earth metal salts, such as calcium and magnesium, or alkali metal salts, such as sodium and potassium salts.

Compounds of the invention are useful because they possess pharmacological activity. Thus, the compounds of the invention are useful as human and animal medicine. They are therefore indicated as pharmaceuticals (and/or in veterinary science), although they may also be used as cosmetics and/or as part of a medical device.

Although compounds of the invention may possess pharmacological activity as such, certain pharmaceutically-acceptable (e.g. “protected”) derivatives of compounds of the invention may exist or may be prepared which may not possess such activity, but which may be administered and thereafter be metabolised or chemically transformed to form compounds of the invention. Such compounds (which may possess some pharmacological activity, provided that such activity is appreciably lower than that of the active compounds to which they are metabolised/transformed) may therefore be described as “prodrugs” of compounds of the invention.

As used herein, references to prodrugs will include compounds that form a compound of the invention, in an experimentally-detectable amount, within a predetermined time, following administration. All prodrugs of the compounds of the invention are included within the scope of the invention.

Compounds of the invention are particularly useful in the treatment of inflammation.

The “treatment of inflammation” includes the treatment of inflammation in any organ of the body (including soft tissue, joints, nerves, the vascular system, internal organs, especially mucosal surfaces, and particularly the skin), irrespective of the cause, and also includes all such inflammatory disorders or conditions, and/or disorders or conditions characterized by inflammation (e.g. as a symptom).

Inflammatory conditions may be (and are typically) characterized by activation of immune defence mechanisms, resulting in an effect that is more harmful than beneficial to the host. Such conditions are generally associated with varying degrees of tissue redness or hyperemia, swelling, edema, hyperthermia, pain (including aching), exudation of body fluids, itching (pruritis), cell death and tissue destruction, cell proliferation, and/or loss of function.

Inflammatory conditions that may be mentioned include arteritis, diabetes mellitus, metabolic syndrome, rosacea, asthma and allergy, ankylosing spondylitis, chronic obstructive pulmonary disease, gouty arthritis, inflammatory bowel disease (such as Crohn's disease and ulcerative colitis), multiple sclerosis, osteoarthritis, pancreatitis, prostatitis, psoriatic arthritis, rheumatoid arthritis, tendinitis, bursitis, Sjogren's syndrome, systemic lupus erythematosus, uveitis, urticaria, vasculitis, mastocytosis, diabetic vascular complications, migraine, atherosclerosis and associated cardiovascular disorders. A disease state characterised by inflammation that may be mentioned is chronic obstructive pulmonary disease (COPD). A further disease state characterised by inflammation that may be mentioned is inflammatory bowel disease, including Crohn's disease and, especially, ulcerative colitis.

Inflammatory conditions that may be more especially mentioned include inflammations of the skin or mucosa (including the oral, nasal, ocular, vaginal, cervical and/or anorectal mucosae, more particularly the oral or nasal mucosae), such as inflammation resulting from infections (such as viral and/or bacterial infections), or allergic/atopic conditions (such as rhinitis, pharyngitis, periodontitis, gingivitis, xerophthalmia, conjunctivitis, dermatitis, urticaria (hives) and food allergy); and other inflammatory conditions, such as herpes, drug eruptions, polymorphous light eruptions, sunburn, early manifestations of skin cancers (erythema-like skin lesions), pathological hair loss (including following skin grafting), chemo rash, psoriasis, erythema multiforme, folliculitis, eczema and external otitis.

More particularly, compounds may be used to treat certain conditions characterised by inflammation, and/or with which inflammation is associated. Such conditions may include wounds (including abrasions (scratches), incisions (including operative incisions), lacerations, punctures, avulsions, bruising and scarring), and burns (including inflammation resulting from surgery following burns, such as skin grafting), and other conditions, such as hemorrhoids.

Wounds of the skin or mucosa may arise from internal or external physical injury to the membrane surface, or may be caused by (i.e. be a symptom of an underlying physiological disorder).

Physical (e.g. “open”) wounds may be caused by sharp objects (cuts, incisions, punctures) or blunt objects/mechanical forces (lacerations, abrasions, avulsions), physical blows (bruises), heat or chemicals (burns and blisters), UV light (sunburn), cold (chilblains or frostbite). Wounds may be superficial (damage only to the epidermis and/or dermis) or may be full thickness wounds (damage below the epidermis and/or dermis). In serious cases, subcutaneous and/or submucosal tissues, such as muscles, bones, joints, and even internal organs, may be damaged.

Compounds of the invention may be used to relieve the pain (including aching) associated with inflammation and/or wounding. In particular, compounds of the invention may be used to relieve procedural pain and/or non-procedural pain. The skilled person will understand that the term “procedural pain” (i.e. operation pain) refers to acute pain that is associated with medical investigations and treatments conducted for the purpose of healthcare. The term “non-procedural” refers to general pain that is associated with inflammation and/or wounding (e.g. pain associated with dental ulcers, burns and/or scars), and is not a consequence of a particular medical intervention.

Compounds of the invention may be used to treat not only the inflammation, pain including aching) and/or pruritis (itching) associated with the wound itself and the healing process, but also they may be used to prevent the exudation of body fluids from wounds, the risk of infection, and also the prevention of physiological reactions that result from inflammation and/or wound healing processes, such as scarring and melanin pigmentation.

Scarring is a consequence of inflammation and/or wound healing and is a general term for the formation of fibrotic tissue that is a consequence of such inflammation/healing.

Compounds of the invention may also be useful in the suppression of the production of melanin pigmentation that may result from inflammation and/or wound healing. Compounds of the invention may also be useful in the suppression of disorders associated with melanin pigmentation, such as chloasma, freckles, melanosis, malar rash and other chromatosis, skin cancers with melanoma, and chromatosis that is caused by exposure to the sun or skin diseases like acne.

Wounds may also arise as a consequence of diseases or disorders. Such wounds may include blistering and/or ulcers of the skin and mucosa. These are common conditions that are often long-lasting and difficult to treat. Skin tissues can often be damaged, removed, liquefied, infected and/or necrotic. Ulcers can lead to secondary consequences to health particularly if they become infected, are hard to heal and are costly. They can also cause significant psychological stress and economic loss to patients, affecting both general well-being and quality of life.

In the alternative, inflammatory skin conditions or diseases in which compounds of the invention find particular utility include psoriasis, acne, eczema and dermatitis, especially allergic/atopic dermatitis, as well as in the treatment of rhinitis, especially allergic rhinitis, hemorrhoids and chronic obstructive pulmonary disease.

Psoriasis is a chronic, inflammatory skin disease with a tendency to recur (some patients never heal during their entire life). Clinical manifestations of psoriasis mainly include erythema and scales. It can occur over the whole body, but is more commonly observed on the scalp and limbs.

Acne is a follicular (pilosebaceous unit) chronic, inflammatory skin disease, the occurrence of which is closely related to main factors like hypersteatosis, blocked pilosebaceous ducts (including closed and open comedones), bacterial infection and inflammatory reactions, that tends to occur during youth, characterised by multiform skin lesions on the face. The term acne thus includes regular acne and acne rosacea (i.e. copper nose).

Eczema is a skin inflammatory reaction with strong itching caused by a variety of internal and external factors. It has three phases, acute, sub-acute, and chronic. In the acute phase, there is a tendency for the production of exudates, while the chronic phase includes infiltration and hypertrophy. Skin lesions are often itchy and recur easily.

Dermatitis is a common skin disease characterised by coarseness, redness, itching, eczema, and dryness. Small lumps, refractory ulcers, and pigmented spots caused by dermatitis may, if not treated promptly, develop to basal cell carcinoma, squamous cell carcinoma, and malignant melanoma. Dermatitis may be caused by various internal and external infectious or non-infectious factors, including substances (contact dermatitis) or allergy (allergic/atopic dermatitis). Also included is seborrheic dermatitis (seborrheic eczema) and all forms of steroid-dependent dermatitis (including light-sensitive seborrheid, perioral dermatitis, rosacea-like dermatitis, steroid-rosacea, steroid-induced rosacea, iatrosacea, steroid dermatitis resembling rosacea, topical corticosteroid-induced rosacea-like dermatitis and, more particularly, facial corticosteroid addictive dermatitis (FCAD) or facial corticosteroid-dependent dermatitis (FCDD), as characterised by flushing, erythema, telangiectasia, atrophy, papules and/or pustules in the facial area after long-term treatment with (including uncontrolled use, abuse or misuse of) topical corticosteroids; see, for example, Xiao et al, J. Dermatol., 42, 697 (2015) and Lu et al, Clin. Exp. Dermatol., 35, 618 (2009)).

Rhinitis is irritation and inflammation of the mucous membrane inside the nose. Common symptoms of rhinitis include a stuffy nose, runny nose, sneezing and post-nasal drip. The most common kind of rhinitis is allergic rhinitis, caused by an allergen, such as pollen, dust, mould, or flakes of skin from certain animals. The compounds of the invention may give rise to relief of eye itchiness, even when administered nasally (i.e. to the nasal mucosa).

Hemorrhoids are swellings caused by larged inflammation of the hemorrhoidal blood vessels found inside or around the rectum and anus. Symptoms include bleeding (i.e. wounding) after the passage of a stool, prolapse of the hemorrhoid, mucus discharge and itchiness, soreness, redness and swelling in the area of the anus. Hemorrhoids are believed to be a consequence of an increase of pressure in the abdomen, for example, as a result of constipation or diarrhea.

Chronic obstructive pulmonary disease (COPD) is the name for a group of lung conditions that cause breathing difficulties, including emphysema (damage to the alveoli) and chronic bronchitis (long-term inflammation of the airways). COPD occurs when the lungs become inflamed, damaged and narrowed. The damage to the lungs is usually irreversible and results in an impairment of the flow of air into and out of the lungs. Symptoms of COPD include breathlessness, productive cough, frequent chest infections and persistent wheezing. The most common cause of the disease is smoking, although other risk factors include high levels of air pollution and occupational exposure to dust, chemicals and fumes.

Compounds of the invention may have positive effects in mitigating erythema, redness and swelling, edema, blisters, and bullous pemphigoid caused by various conditions including those mentioned generally and specifically herein, and may inhibit exudation of subcutaneous tissue fluid, and suppressing itching and pain caused by such inflammatory conditions.

Other inflammatory conditions that may be mentioned include:
(a) Mucosal inflammation, such as oral mucositis, aphthous ulcers, otitis media, laryngitis, tracheitis, esophagitis, gastritis, enteritis and enterocolitis (including bacillary dysentery, chronic amoebic dysentery, schistosomiasis, nonspecific ulcerative colitis and regional enteritis), cervicitis and endocervicitis, endometritis, inflammation caused by inhalation injury and the like, as well as mucosal inflammation associated with cancers, and infections (e.g. viral infections, such as the common cold or influenza), that affect mucosal surfaces, such as those in the oral cavity, the nasopharynx, the ear, the throat, the trachea, the gastrointestinal tract, the cervix, etc.
(b) Orthopedic inflammation associated with, for example bone fractures, pyogenic infection of bones and joints, inflammation caused by rheumatic bone diseases, as well as pyogenic osteomyelitis (acute, chronic, localized, sclerotic, post-traumatic), pyogenic arthritis; bone tumors (osteoma, osteoid osteoma, chondroma), bone cysts, osteoclastoma, primary bone sarcoma (osteosarcoma, chondrosarcoma, osteofibrosarcoma, Ewing's sarcoma, non-Hodgkin's lymphoma, myeloma, chordoma), metastatic bone tumors, tumor-like lesions of bone (bone cyst, aneurysmal bone cyst, eosinophilic granuloma, fibrous dysplasia); and rheumatic arthritis.
(c) Nerve inflammation, such as peripheral polyneuritis, facial neuritis, peripheral neuritis, subcutaneous neuritis, ulnar neuritis, intercostal neuritis, etc.
(d) Subcutaneous and submucosal soft tissue inflammation, such as myositis, ligamentitis, tendonitis, panniculitis capsulitis, lymphadenitis, bubonadentitis, tonsillitis, synovitis, fasciitis, and soft tissue inflammation caused by injuries, contusion or laceration of muscles, ligaments, fascia, tendons, membrana synovialis, fat, articular capsules, and lymphoid tissue.
(e) Vascular inflammation, such as allergic leukocytoclastic vasculitis, allergic cutaneous vasculitis, polyarteritis nodosa, thrombotic vasculitis, granulomatous vasculitis, lymphocytic vasculitis, vasculitis with abnormalities in blood composition, and rheumatic vasculitis, as well as vascular inflammation associated with vascular cancers caused by allergic leukocytoclastic vasculitis, polyarteritis nodosa, thrombotic vasculitis, granulomatous vasculitis, lymphocytic vasculitis, vasculitis with abnormalities in blood composition, and rheumatic vasculitis.
(f) Inflammation of the internal organs, such as the heart, stomach, intestine, lung, liver, spleen, kidney, pancreas, bladder, ovary, and prostate, including but not limited to pericarditis, myocarditis, endocarditis, pneumonia, hepatitis, splenitis, nephritis pancreatitis, cystitis, oophoritis, prostatitis and treatment of gastric ulcer.
(g) Inflammation of the eye and surrounding area, such as conjunctivitus, keratitis (e.g. acute epithelial keratitis, nummular keratitis, interstitial keratitis, disciform keratitis, neurotrophic keratitis, mucous plaque keratitis, herpes simplex keratitis, herpes zoster keratitis, bacterial keratitis, fungal keratitis acanthamoebic keratitis, onchocercal keratitis, superficial punctate keratitis, ulcerative keratitis, exposure keratitis photokeratitis and ccontact lens acute red eye), optic neuritis, etc.
(h) Inflammation of the gums and the oral cavity, such as periodontitis, gingivitis, dental ulcers, etc.
(i) Inflammation associated with rheumatism, such as rheumatic vasculitis, rheumatoid arthritis, rheumatic bone diseases, ankylosing spondylitis, bursitis, Crohn's disease, gout, infectious arthritis, juvenile idiopathic arthritis, osteoarthritis, osteoporosis, polymyalgia rheumatica, polymyositis, psoriatic arthritis, scleroderma, Sjogren's syndrome, spondyloarthropathies, systemic lupus erythematosus, tendinitis, etc.

Compounds of the invention may also be used in the treatment of certain specific diseases of the respiratory system, such as pulmonary cystic fibrosis, usual interstitial pneumonia, allergic pneumonia, asbestosis, emphysema, pulmonary heart disease, pulmonary embolism, etc. A specific disease state that may be mentioned in idiopathic pulmonary fibrosis.

Idiopathic pulmonary fibrosis is a diffuse and fatal pulmonary interstitial disease with pathological features including alveolar epithelial damage, massive proliferation of lung fibroblasts, excessive deposition of extracellular matrix, ultimately leading to irreversible lung tissue damage. In the latter stages of the disease, subjects with idiopathic pulmonary fibrosis experience respiratory failure and death. It has been found that compounds of the invention may find utility in the treatment of idiopathic pulmonary fibrosis and/or alleviation of the symptoms associated with the disease.

Compounds of the invention may further possess an antioxidation effect, by increasing SOD (superoxide dismutase) production and reducing lipid oxidation. Such compounds and formulations including them may therefore be considered have antioxidant properties.

Compounds of the invention may also possess antipyretic properties that allow for the treatment of a fever and/or alleviate the symptoms thereof; for example, by reducing a subject's body temperature, which results in a reduction of fever. Compounds of the invention and formulations including them may therefore be considered to be antipyretics.

According to a further aspect of the invention there is provided a method of treatment of inflammation, of an inflammatory disorder, and/or of a disorder/condition characterised by inflammation (for example as a symptom), which method comprises the administration of a compound of the invention to a patient in need of such treatment.

For the avoidance of doubt, in the context of the present invention, the terms “treatment”, “therapy” and “therapy method” include the therapeutic, or palliative, treatment of patients in need of, as well as the prophylactic treatment and/or diagnosis of patients which are susceptible to, inflammation and/or inflammatory disorders.

“Patients” include reptilian, avian and mammalian (particularly human) patients.

In accordance with the invention, compounds of the invention are preferably administered locally or systemically, for example orally, intravenously or intraarterially (including by intravascular and other perivascular devices/dosage forms (e.g. stents)), intramuscularly, cutaneously, subcutaneously, transmucosally (e.g. sublingually or buccally), rectally, intravaginally, transdermally, nasally, pulmonarily (e.g. tracheally or bronchially), preferably topically, or by any other parenteral route, in the form of a pharmaceutical preparation comprising the compound(s) in pharmaceutically acceptable dosage form(s). Administration by inhalation (e.g. nasally) is particularly useful when the condition to be treated is rhinitis or inflammation resulting from viral infections of the airways (e.g. common cold, influenza). Pulmonary administration is particularly useful when the condition to be treated is COPD or IPF. Topical forms of administration may be enhanced by creating a spray comprising active ingredients, e.g. by using a powder aerosol or by way of an aqueous mist using an appropriate atomisation technique or apparatus, such as a nebulizer.

Preferred modes of delivery of compounds of the invention include topically to the site of inflammation (e.g. the mucosa, including the lung or, more preferably, the skin) in an appropriate (for example pharmaceutically- and topically-acceptable) vehicle suitable for application to the skin and/or the appropriate mucosal surface, and/or a commercially-available formulation, but may also include oral, intravenous, cutaneous or subcutaneous, nasal, intramuscular, intraperitoneal or pulmonary delivery.

Compounds of the invention will generally be administered in the form of one or more for example pharmaceutical formulations in admixture with a (e.g. pharmaceutically acceptable) adjuvant, diluent or carrier, which may be selected with due regard to the intended route of administration (e.g. topical to the relevant mucosa (including the lung) or, preferably, the skin) and standard pharmaceutical or other (e.g. cosmetic) practice. Such pharmaceutically acceptable carriers may be chemically inert to the active compounds and may have no detrimental side effects or toxicity under the conditions of use. Such pharmaceutically acceptable carriers may also impart an immediate, or a modified, release of the active ingredient.

Suitable pharmaceutical formulations may be commercially available or otherwise prepared according to techniques that are described in the literature, for example, Remington The Science and Practice of Pharmacy, 22^{nd} edition, Pharmaceutical Press (2012) and Martindale—The Complete Drug Reference, 38^{th} Edition, Pharmaceutical Press (2014) and the documents referred to therein, the relevant disclosures in all of which documents are hereby incorporated by reference. Otherwise, the preparation of suitable formulations including compounds of the invention may be achieved non-inventively by the skilled person using routine techniques.

Compounds of the invention may be in the form of an aqueous formulation such as an emulsion, a suspension and/or a solution (e.g. an (optionally) buffered aqueous formulation, such as a physiological saline-containing formulation, a phosphate-containing formulation, an acetate-containing formulation or a borate-containing formulation), or a freeze-dried powder.

Active ingredient may further be combined with appropriate excipents to prepare:
gel formulations (for which suitable gel matrix materials include cellulose derivatives, carbomer and alginates, gummi tragacanthae, gelatin, pectin, carrageenan, gellan gum, starch, Xanthan gum, cationic guar gum, agar, noncellulosic polysaccharides, saccharides such as glucose, glycerin, propanediol, vinyl polymers, acrylic resins, polyvinyl alcohol, carboxyvinyl polymer and, particularly, hyaluronic acid);
lotions (for which suitable matrix materials include cellulose derivatives, glycerin, noncellulosic polysaccharides, polyethylene glycols of different molecular weights and propanediol);
pastes or ointments (for which suitable paste matrix materials include glycerin, vaseline, paraffin, polyethylene glycols of different molecular weights, etc.);
creams or foams (for which suitable excipients (e.g. foaming agents) include hydroxypropyl methyl cellulose, gelatin, polyethylene glycols of different molecular weights, sodium dodecyl sulfate, sodium fatty alcohol polyoxyethylene ether sulfonate, corn gluten powder and acrylamide);
powder aerosols (for which suitable excipients include mannitol, glycine, dextrin, dextrose, sucrose, lactose, sorbitol and polysorbates, e.g. a dry powder inhalant); and/or
liquid (aerosol) sprays for oral use or for inhalation (for which suitable excipients include viscosity modifiers, such as hyaluronic acid, sugars, such as glucose and lactose, emulsifiers, buffering agents, alcohols, water, preservatives, sweeteners, flavours, etc.).

Moisturizing agents, such as glycerol, glycerin, polyethylene glycol, trehalose, glycerol, petrolatum, paraffin oil, silicone oil, hyaluronic acid and salts (e.g sodium and potassium salts) thereof, octanoic/capyic triglyceride, and the like; and/or antioxidants, such as vitamins and glutathione; and/or pH modifiers, such as acids, bases and pH buffers, may also be included in such formulations, as appropriate. Furthermore, surfactants/emulsifiers, such as hexadecanol (cetyl alcohol), fatty acids (e.g. stearic acid), sodium dodecyl sulfate (sodium lauryl sulfate), sorbitan esters (e.g. sorbitan stearate, sorbitan oleate, etc.), monoacyl glycerides (such as glyceryl monostearate) polyethoxylated alcohols, polyvinyl alcohols, polyol esters, polyoxyethylene alkyl ethers (e.g. polyoxyethylene sorbitan monooleate), polyoxyethylene castor oil derivatives, ethoxylated fatty acid esters, polyoxylglycerides, lauryl dimethyl amine oxide, bile salts (e.g. sodium deoxycholate, sodium cholate), phospholipids, N,N-dimethyldodecylamine-N-oxide, hexadecyltrimethyl-ammonium bromide, poloxamers, lecithin, sterols (e.g. cholesterol), sugar esters, polysorbates, and the like; preservatives, such as phenoxyethanol, ethylhexyl glycerin, and the like; and thickeners, such as acryloyldimethyltaurate/VP copolymer, may be included. In particular stearic acid, glyceryl monostearate, hexadecanol, sorbitan stearate, cetyl alcohol, octanoic/capric glyceride etc. may be included, particularly in cream formulations.

Compounds of the invention and (e.g. solutions, gels, creams, ointments, lotions, foams, pastes and/or dry powders as described above including them), may further be combined with an appropriate matrix material to prepare a dressing or a therapeutic patch for application on a biological surface, such as the skin or a mucosal surface. Such formulations may thus be employed to impregnate a matrix material, such as gauze, non-woven cloth or silk paper. The therapeutic patch may alternatively be, for example, a band-aid, a facial mask, an eye mask, a hand mask, a foot mask, etc.

Vaseline may be employed for use in applying such dressings to wounds, but we have also found that ointments based on PEGs (e.g. PEG 400) may be combined with matrix materials to prepare dressings without the need to use vaseline.

Compounds of the invention may also be combined in treatment with one or more growth factors selected from platelet-type growth factors (including platelet-derived growth factors, PDGFs); osteosarcoma-derived growth factors (ODGF), epidermal growth factors (EGFs), transforming growth factors (TGFα and TGFβ), fibroblast growth factors (aFGF, BFGF), insulin-like growth factors (IGF-I, IGF-II), nerve growth factors (NGF), interleukin-type growth factors (IL-1, IL-1, IL-3), erythropoietin (EPO), and colony stimulating factor (CSF).

According to a further aspect of the invention there is provided a (e.g. pharmaceutical) composition comprising a compound of the invention and one or more pharmaceutically-acceptable excipient, such as an adjuvant, diluent or carrier. Preferred formulations are suitable for application locally to e.g. the mucosa (including the lung) or, more preferably, the skin and therefore comprise a topically-acceptable adjuvant, diluent or carrier.

Thus formulations comprising compounds of the invention may be suitable for, adapted for, and/or packaged and presented for topical administration (e.g. to the lung, the mucosa or to the skin), for example for the treatment of a disorder, including inflammation, an inflammatory disorder and/or a condition characterised by inflammation (e.g. as a symptom) by way of direct topical administration of that formulation (e.g. to the lung, the mucosa or, preferably, to the skin).

In relation to this aspect of the invention, for the avoidance of doubt, topical formulations of the invention may be used in any and all conditions described herein, including treatments of inflammation, in the treatment of any and all inflammatory disorder(s), and/or in the treatment of any and all condition(s) characterised by inflammation, as hereinbefore mentioned, defined or described.

Topical (e.g. liquid- or (e.g. aqueous) solution-based) formulations of the invention may be particularly useful in wound recovery, and may alleviate pain (including aching) and, particularly, pruritis/itching that is associated with the wound itself and the wound healing process. Such topical formulations of the invention may be particularly useful in the prevention and/or suppression of the exudation of body fluids from wounds, particularly during the acute inflammation stage, for example during the first 48 hours, after a burn or wound has been inflicted. This prevents the risk of infection, and other physiological reactions. Such topical formulations of the invention may also be particularly useful in the prevention and/or suppression of scarring and melanin pigmentation (vide supra), whether associated with wounds or otherwise.

Administration of active ingredients may be continuous or intermittent. The mode of administration may also be determined by the timing and frequency of administration, but is also dependent, in the case of the therapeutic treatment of inflammation, on the severity of the condition.

Depending on the disorder, and the patient, to be treated, as well as the route of administration, compounds of the invention may be administered at varying therapeutically effective doses to a patient in need thereof.

Similarly, the amount of active ingredient in a formulation will depend on the severity of the condition, and on the patient, to be treated, but may be determined by the skilled person.

In any event, the medical practitioner, or other skilled person, will be able to determine routinely the actual dosage, which will be most suitable for an individual patient, depending on the severity of the condition and route of administration. The dosages mentioned herein are exemplary of the average case; there can, of course, be individual instances where higher or lower dosage ranges are merited, and such are within the scope of this invention.

Doses may be administered between once and four times daily.

Appropriate concentrations of compounds of the invention in an aqueous solution product may be about 0.01 (e.g. about 0.1) to about 15.0 mg/mL, in all cases calculated as the free (non-salt) peptide.

Appropriate topical doses of compounds of the invention are in the range of about 0.05 to about 50 μg/cm² of treated area, such as about 0.1 (e.g. about 0.5) to about 20 μg/cm² of treated area, including about 1 to about 10 μg/cm²) of treated area, such as about 5 μg/cm² of treated area, in all cases calculated as the free (non-salt) peptide.

We prefer that pH values of formulations comprising compounds of the invention are in the range of about 1.0 to about 9.0 (for example about 3.0 to about 8.0). However, we have found that compounds of the invention are considerable more stable at all pH values, including neutral and basic pHs, than for example MAPs, and isolated compounds that consist of the mefp-1 decapeptide sequence.

In any event, the dose administered to a mammal, particularly a human, in the context of the present invention should be sufficient to effect a therapeutic response in the mammal over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

In the uses and methods described herein, compounds of the invention may also be combined with one or more active ingredients that are useful in the treatment of inflammation and/or inflammatory disorders (other antiinflammatory agents). Such patients may thus also (and/or already) be receiving therapy based upon administration of one or more of such other active ingredients, by which we mean receiving a prescribed dose of one or more of those active ingredients mentioned herein, prior to, in addition to, and/or following, treatment with a compound of the invention.

Such anti-inflammatory agents that may be used in combination with compounds of the invention in the treatment of inflammation include therapeutic agents that are useful in the treatment of inflammation and/or of diseases characterised by inflammation as one of its symptoms. Depending on the condition to be treated, such antiinflammatory agents may include NSAIDs, leukotriene receptor antagonists (e.g. montelukast, for example as described hereinafter), corticosteroids, analgesics and certain enzymes, such as trypsin, for example as described hereinafter. Compounds of the invention may also be combined with leukotriene B4 (LTB4).

In this context, compounds of the invention may also be combined for use in the treatment of inflammation with one or more mussel adhesive proteins (MAPs), which includes any adhesive protein that may be derived from mussel species, such as Mytilus edulis (blue mussel), including full length proteins, including all sub-types, that are or may be derived from mussels, such as the collagens pre-COL-P, pre-COL-D and pre-COL-NG, the mussel feet matrix proteins PTMP and DTMP, and, more preferably, mfps or mefps, such as mefp-2, mefp-3, mefp-4, mefp-5, mefp-6 and especially mefp-1, and includes mixtures or combinations of any of these proteins, such as mefps. Although mixtures/combinations of the aforementioned MAP sub-types may be provided in accordance with the invention, we prefer that the purity of the principal MAP sub-type (e.g. mefp-1) is at least 25% by weight of the total amount of any such mixture.

Naturally-occurring MAPs may be prepared, for example by mixed adsorption chromatography (see Chinese Patent No. ZL200710179491.0), by carboxymethyl ion exchange chromatography (see Chinese Patent No. ZL200710179492.5), and/or by salting out and dialysis (Chinese Patent No. ZL200910087567.6). Commericial sources of MAPs include USUN Bio Co. (China; sold as MAP Medical Device®), BD Biosciences (USA), Kollodis (South Korea) and Biopolymer (Sweden). MAPs may alternatively be produced using known recombinant DNA methods.

Derivatives (e.g. pharmaceutically-acceptable derivatives) of MAPs may also be combined with compounds of the invention and include compounds with, for example, molecular weights in the range of about 500 to about 2,000 (e.g. about 1,500, such as about 1,200 including about 800 Da) which may allow for easier penetration through biological membranes, such as the skin barrier or a mucosal surface. Such derivatives may also include other compounds that comprise amino acid sequences that are the same as, or are (e.g. minor) variants of, sequences that have been identified in naturally-occurring MAPs, and which may be synthesized by chemical and/or biological processes (e.g. chemical modifications of naturally-occurring MAPs, or direct synthesis). By “(e.g. minor) variants of amino acid sequences identified in naturally-occurring MAPs”, we mean variations in those sequences that do not negatively affect the requisite properties of the relevant naturally-occurring MAP to a measurable degree.

For example, as discussed hereinbefore, the isolated decapeptide compound of the sequence: Ala-Lys-Pro-Ser-Tyr-Hyp-Hyp-Thr-DOPA-Lys (SEQ ID NO: 1, mefp-1 decapeptide) is a pharmaceutically-acceptable low molecular weight derivative of MAP that may be combined with a compound of the invention.

Such derivatives of MAPs may be employed in combination products according to the invention alone, or in combination with one or more other such derivatives, and/or one or more of the aforementioned full length MAPs.

Appropriate concentrations of MAPs and derivatives thereof in an aqueous solution topical formulation product may be about 0.01 (e.g. about 0.1) to about 15.0 (e.g. about 1.5) mg/mL, and appropriate pH values are in the range of about 1.0 to about 7.0 (for example about 3.0 to about 6.5), irrespective of whether the formulation employed is a combined preparation or a kit of parts as hereinbefore described. Suitable commercial sources of such aqueous solutions include USUN Bio Co., Jiangyin, Jiangsu Province, China.

Appropriate topical doses of MAPs and derivatives thereof are in the range of about 0.1 to about 50 μg/cm² of treated area, such as about 1 to about 20 μg/cm² of treated area, including about 2 to about 10 μg/cm² of treated area, such as about 5 μg/cm² of treated area.

Combinations of compounds of the invention and mefp-1 have been found to be of particular utility in the treatment of inflammatory bowel diseases, such as ulcerative colitis and, in particular, in arresting the bleeding in patients having such a disease.

Other preferred agents that may be combined with compounds of the invention include LTB4 (to treat wounds and burns), montelukast (to treat inflammation generally) and trypsin (to treat inflammation of the mucosa associated with e.g. viral infections).

Compounds of the invention may also be combined with other therapeutic agents which, when administered, are known to give rise to inflammation as a side-effect.

When compounds of the invention may be “combined” with other therapeutic agents in this way, the active ingredients may be administered together in the same formulation, or administered separately (simultaneously or sequentially) in different formulations.

Such combination products provide for the administration of compounds of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:
(1) a pharmaceutical formulation including a compound of the invention; another antiinflammatory agent, or agent known to give rise to inflammation as a side-effect; and a pharmaceutically-acceptable adjuvant, diluent or carrier (which formulation is hereinafter referred to as a “combined preparation”); and
(2) a kit of parts comprising components:
(A) a pharmaceutical formulation including a compound of the invention in admixture with a pharmaceutically-acceptable adjuvant, diluent or carrier; and
(B) a pharmaceutical formulation including another antiinflammatory agent, or agent known to give rise to inflammation as a side-effect, in admixture with a pharmaceutically-acceptable adjuvant, diluent or carrier,
   which components (A) and (B) are each provided in a form that is suitable for administration in conjunction with the other.

According to a further aspect of the invention, there is provided a method of making a kit of parts as defined above, which method comprises bringing component (A), as defined above, into association with a component (B), as defined above, thus rendering the two components suitable for administration in conjunction with each other.

By bringing the two components “into association with” each other, we include that components (A) and (B) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:
(I) one of components (A) and (B) as defined herein; together with
(II) instructions to use that component in conjunction with the other of the two components.

The kits of parts described herein may comprise more than one formulation including an appropriate quantity/dose of a compound of the invention, and/or more than one formulation including an appropriate quantity/dose of another antiinflammatory agent, in order to provide for repeat dosing. If more than one formulation (comprising either active compound) is present, such formulations may be the same, or may be different in terms of the dose of either compound, chemical composition(s) and/or physical form(s).

With respect to the kits of parts as described herein, by “administration in conjunction with”, we include that respective formulations comprising a compound of the invention and other antiinflammatory agent are administered, sequentially, separately and/or simultaneously, over the course of treatment of the relevant condition.

Thus, in respect of the combination product according to the invention, the term “administration in conjunction with” includes that the two components of the combination product (compound of the invention and other antiinflammatory agent) are administered (optionally repeatedly), either together, or sufficiently closely in time, to enable a beneficial effect for the patient, that is greater, over the course of the treatment of the relevant condition, than if either a formulation comprising compound of the invention, or a formulation comprising the other agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration of the other component. When used in this context, the terms “administered simultaneously” and “administered at the same time as” include that individual doses of the relevant compound of the invention and other antiinflammatory agent are administered within 48 hours (e.g. 24 hours) of each other.

In a further aspect of the invention, there is provided a process for the preparation of a combined preparation as hereinbefore defined, which process comprises bringing into association a compound of the invention, the other antiinflammatory agent, or agent known to give rise to inflammation as a side-effect, and at least one (e.g. pharmaceutically-acceptable) excipient.

Wherever the word “about” is employed herein, for example in the context of amounts, such as concentrations and/or doses of active ingredients, molecular weights or pHs, it will be appreciated that such variables are approximate and as such may vary by +10%, for example+5% and preferably +2% (e.g. +1%) from the numbers specified herein. In this respect, the term “about 10%” means e.g. +10% about the number 10, i.e. between 9% and 11%.

Compounds of the invention have the advantage that they may be used in variety of conditions characterised by inflammation, whether that condition is an organic inflammatory disease per se or is associated with, or is characterised by, inflammation (e.g. a wound, a burn or a viral infection).

Compounds of the invention also have the advantage in that they are physico-chemically stable to processes such as oxidation at a variety of pHs, including neutral and basic pHs, when compared to similar compounds known in the prior art, such as MAPs and isolated mefp-1 decapeptide.

The uses and methods described herein may also have the advantage that, in the treatment of the conditions mentioned hereinbefore, they may be more convenient for the physician and/or patient than, be more efficacious than, be less toxic than, have a broader range of activity than, be more potent than, produce fewer side effects than, or that it/they may have other useful pharmacological properties over, similar methods (treatments) known in the prior art, whether for use in the treatment of inflammation, inflammatory disorders, or disorders characterised by inflammation as a symptom (including wounds), or otherwise.

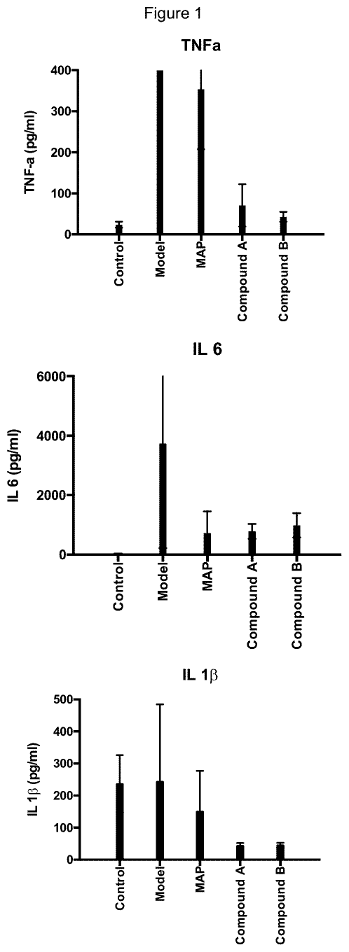

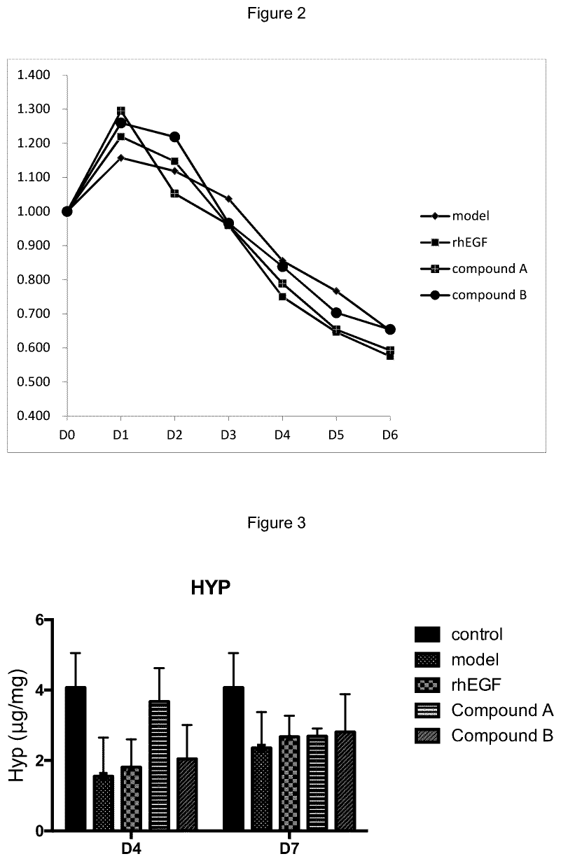

The invention is illustrated by the following examples, in which, in a mouse wound model, FIG. 1 shows ELISA test results for various inflammatory markers obtained from exudates from air pouches induced in mice according to Example 1, tested with various test compounds; FIG. 2 shows the effect on acute healing of wounds inflicted on mice when treated with various test compounds; and FIGS. 3 to 5 shows ELISA test results for inflammatory markers (Hyp, VEGF and TNF-β1, respectively) obtained from samples taken from the respective wounds in those mice.

### EXAMPLES

### Example 1

### Synthesis of Ala-Lys-Pro-Ser-Tyr-Hyp-Hyp-Thr-Tyr-Lys (SEQ ID NO: 2)

Fmoc-Lys-Boc-Wang resin (0.3 mmol/g; GLS180322-41301, GL Biochem, Shanghai, China) was loaded into a reaction column.

2 litres of methylene chloride (DCM; Shandong Jinling Chemical Industry Inc. Co., Shandong, China) was added to the column and allowed to soak the resin for about half an hour. The DCM was then drawn off and 2 litres of N,N-dimethylformamide (DMF; Shandong Shitaifeng Fertilizer Industry Inc. Co., Shandong, China) was used to wash the column three times.

200 mL of piperidine (Shanghai Li Ming Industry and Trade Co., Ltd., China] was mixed with 1 litre of DMF and was used as deprotection solution. The liquid was drained after 15 minutes and the column was washed with DMF six times.

69 g of Fmoc-Tyr (tBu)-OH (GLS170916-36901, GL Biochem) and 48 g of 2-(1H-benzotriazole-1-yl)-1,1,3,3-tetramethylaminium tetrafluoroborate (TBTU; GL Biochem) were dissolved in 300 mL of DMF and were added to the reaction column. 53 mL of N,N-diisopropylethylamine (DIPEA; Suzhou Highfine Biotech Co. Ltd, Jiangsu, China) was then added. The reaction time was one hour.

A sample was taken, and ninhydrin (Shanghai Shanpu Chemical Co. Ltd, China) was used to detect when the reaction was complete. At this point, the liquid was drained and the residue was washed three times with DMF.

The above coupling steps were repeated to couple the remaining amino acids in the same amounts: Fmoc-Thr (tBu)-OH, Fmoc-4-Hyp (tBu)-OH, Fmoc-4-Hyp (tBu)-OH, Fmoc-Tyr (tBu)-OH, Fmoc-Ser (tBu)-OH, Fmoc-Pro-OH, Fmoc-Lys (Boc)-OH and Fmoc-Ala-OH.

At the end of the reaction sequence, 20% piperidine in DMF was added as a deprotection solution in the manner described above. The liquid was then drained after 15 minutes and the column washed with DMF, DCM and methanol, 3 times each, respectively.

The liquid was drained to obtain the resin-bound polypeptide.

An appropriate amount of lysate, which was comprised of 95% trifluoroacetic acid (TFA), 2.5% water and 2.5% triisopropylsilane (Tis), was added to immerse the resin-bounded polypeptide. The mixture was put on a shaker and incubated for 2 hours at 30 to 35° C. The resin was then removed by filtration.

Anhydrous ice ether was added into the filtrate, which was centrifuged and the supernatant discarded. The resultant was washed with anhydrous ice ether three times. The isolated peptide was dried by desiccation to obtain 128 g of crude polypeptide. The crude compound was desalted using an anion exchange resin, analysed and freeze-dried. Approximately 70.7 g of purified peptide was obtained after purification, which was re-tested for confirmation.

1 mg of crude product was dissolved in 1 mL of an acetonitrile and water mixture (1:3) and detected using a P3000A HPLC pump and LC3000 semi-preparation equipment (preparation column model: GS-120-10-C18-AP 30 mm; Beijing Chuangxintongheng Science & Technology Co., Ltd., Beijing, China). The appropriate gradient for elution was calculated and the target peak was detected at 14.351 min with LCMS (analysis column model: GS-120-5-C18-BIO, 4.6*250 mm; detection: UV at 220 nm; solvent A: 0.1% TFA in MeCN, solvent A: 0.1% TFA in water; flow rate 1.0 mL/min.; volume: 10 μL).

m/z 592.65 [M+2H]²⁺ (97.89%).

### Example 2

### Air Pouch Model

Healthy adult male C57BL/6 mice weighing between 20 and 30 g were supplied by Nanjing Biomedicial Research Institute of Nanjing University (NBRI). Prior to any experiments being conducted, mice were housed under standardized conditions (at a constant temperature of 22±2° C., with alternating 12 hour periods of light and darkness), and were fed on a standard mouse diet with water, for about a week.

General anesthesia was induced using intraperitoneal 3% chloral hydrate (Sinopharm Chemical Reagent Co., Ltd., Shanghai, China; 1 mL/10 g of body weight). The hair of the entire dorsum was shaved and depilated 1 day before sterile air injection.

Air pouches were produced by subcutaneous injection of sterile air (5 mL) into the intrascapular area of the mice. After three days, another injection of air (3 mL) was performed to maintain the pouches. In order to induce acute inflammation, three days after this final injection, animals received an injection of sterile carrageenan solution (CP Kelco, Taixing, Jiangsu Province, China. 1%, 0.5 mL; produced by adding 0.1 g of carrageenan powder into a beaker containing 10 mL of 0.9% saline solution and stirring). Mice were pre-treated with test samples or vehicle 1 hour before and 23 hours after the carrageenan injection into the subcutaneous air pouch. Animals were sacrificed 24 hours after the carrageenan injection.

Skin biopsies were taken from the air pouches. A part of the biopsy was fixed in formalin (produced by adding ultra pure water to 50 mL of a 40% formaldehyde solution (Nanchang Rain Dew Experimental Equipment Co., Ltd., Nanchang, Hubei Province, China) up to a total volume of 500 mL) and analyzed by histological embedding in paraffin wax, sectioning and staining.

The cavity was washed with 4 mL of sterile phosphate buffer solution (pH 7.4; prepared by dissolving 4 g of NaCl, 0.1 g of KCl, 1.749 g of Na₂HPO₄·12H₂O and 0.1 g of KH₂PO₄ in ultrapure water, adjusting the pH to 7.4 with HCl and diluting with water to a total volume of 500 mL).

Exudates were collected and volumes were quantified. The exudates were centrifuged at 3000 rpm for 10 minutes at 4° C., and the supernatants were collected and stored at −20° C. for ELISA analysis using standard ELISA test kits from Beijing 4A Biotech Co., Ltd. (Beijing) and an ELISA reader (SH-1000 Hitachi, Japan) for tissue necrosis factor alpha (TNF-α), interleukin 1 beta (IL-1B), interleukin 6 (IL-6).

After conducting some preliminary experiments to validate the model, an experiment was conducted in which mice were treated by administering test samples or vehicle in accordance with Table 1 below.

In Table 1, MAP solution was prepared as follows. Blue mussels were harvested in the coastal area of Shandong Province, China. Mussel feet were collected, cut into small pieces and homogenized in an extraction buffer comprising 5% acetic acid in 4 mol/L of aqueous urea. The crude extracts were collected after centrifuging and then purified by liquid chromatography. The purified protein (semi-finished product; concentration 8 mg/mL; purity as measured by HPLC 91.72%; pH 4.2) was stored at 0° C. The solutions that was employed below was prepared by adding saline solution to this semi-finished product to obtain the concentrations described in Table 1.

An isolated compound consisting of the mefp-1 decapeptide sequence Ala-Lys-Pro-Ser-Tyr-Hyp-Hyp-Thr-DOPA-Lys (SEQ ID NO: 1, hereinafter “Compound A”) was purchased from Innovagen (Innovagen AB, Lund, Sweden). It was stored as a powder at

−20° C. and dissolved in saline water at a concentration of 0.6 mg/ml, pH 5.5. 0.5 mL of the solution was injected.

The compound of the invention (Compound B) was synthesized by GL Biochem (Shanghai) Ltd. according to Example 1 above, and was also stored as a powder at −20° C. and was dissolved in saline water at a concentration of 0.6 mg/ml, pH 5.5. 0.5 mL of the solution was injected.

All the substances listed in Table 1 were administrated topically by directly injecting into the air pouches.

TABLE 1

| | | | | Timing of |
|---|---|---|---|---|
| | | | | Treatment |
| | | | | (before |
| | No. | Drug concn. | | carrageenan |
| Group | Mice | (mg/mL) | Dose/mouse | injection) |
| Control (air | 5 | Normal | 4.5 mg (of NaCl) | n/a |
| injection only) | | saline | | |
| Model | 5 | Normal | 4.5 mg (of NaCl) | 1 hour |
| (carrageenan | | saline | | |
| injection) | | | | |
| MAP | 5 | 0.6 | 300 μg | 1 hour |
| Compound A | 5 | 0.6 | 300 μg | 1 hour |
| Compound B | 5 | 0.6 | 300 μg | 1 hour |

The histological specimens were analyzed and an inflammation score, an activity score (i.e. the number and density of neutrophils shown in pathological slides, indicating the degree of inflammation and, in the case of open wound and infective diseases, the degree of infection), an edema score and a fibroblast proliferation score were estimated as follows.

The HE stained slices were observed under an optical microscope and were scored (1, 2 or 3 points) according to the perceived inflammation level (in cases showing only a small amount of inflammatory cells scattered in the area-1 point (mild); in cases where many inflammatory cells were observed-2 points (moderate); and, in cases with diffuse infiltration-3 points (severe). A similar scoring system was employed for edema levels (3 points for most severe and 1 point for mild) after overall observation. Scores for neutrophils employed the same methodology as that employed for inflammatory cells.

TABLE 2

| | | | Fibroplastic | |
|---|---|---|---|---|
| | Inflammation | Edema | proliferation | |
| Group | score | Score | score | Sum |
| Control | 1 | 1.2 | 0.8 | 2.2 |
| Model | 2.2 | 1.8 | 0.6 | 4 |
| MAP | 2 | 1.2 | 0.4 | 3.2 |
| Compound A | 1.6 | 1.8 | 0.2 | 3.4 |
| Compound B | 1.6 | 1.2 | 1.00 | 2.8 |

The ELISA test results of the exudate for TNF-α, IL-6 and IL-1B are shown graphically in FIG. 1. The results showed that Compound B had very strong anti-inflammatory effects.

### Example 3

### Acute Wound Model

6-8 weeks old male C57BL/6 mice were supplied by Changzhou Cvens Experimental Animal Co. Ltd (Changzhou, Jiangsu Province, China). Prior to any experiments being conducted, mice were housed under standardized conditions (at a constant temperature of 22±2° C., with alternating 12 hour periods of light and darkness), and were fed on a standard mouse diet with water, for about a week.

General anesthesia was induced using intraperitoneal 3% chloral hydrate (1 mL/10 g of body weight). The hair on the back was shaved by a baby hair shaver and depilated with cream. The skin area was wiped and sterilized with 75% alcohol twice.

An EMS skin biopsy punch (Electron Microscopy Sciences, P.O. Box 550, 1560 Industry Road, Hatfield, PA, USA) with a 18 mm diameter was used to make a round wound on the back. The full thickness of skin was removed and the depth of the wound reached the fascia. Wounds were left open without a suture.

Different drugs were administrated topically at 50 μL/wound, once daily from Day 0 to Day 12, as shown in Table 3 below. The control group did not have a wound inflicted. The model group was given same amount of normal saline. There were 8 mice in each group.

Recombinant Human Epidermal Growth Factor (rhEGF, Shanghai Haohai Biological Technology Co. Ltd, 23/F, Shanghai, China) was purchased and prepared according to the manufacturer's instructions. The lyophilized rhEGF powder (100000 IU/vial) was dissolved in 20 mL of normal saline to make a solution with a concentration of 5000 IU/mL. The working dose of rhEGF for this experiment was 1285 IU/wound.

For this experiment, Compound A was also obtained from GL Biochem (Shanghai) Ltd. The peptide powders were stored at −20° C. and dissolved in saline at concentrations of 61.8 μg/mL (Compound A), and 61 μg/mL (Compound B). 50 μL of the solutions were applied onto the wound surfaces.

TABLE 3

| GROUP | SAMPLE | DOSAGE | |
|---|---|---|---|
| Control | Saline | / | |
| Model | Model + saline | / | |
| rhEGF | Model + rhEGF | 1285 | IU/mouse |
| Compound A | Model + Compound A | 3.09 | μg/mouse |
| Compound B | Model + Compound B | 3.05 | μg/mouse |

Wounds were dressed with gauze and transparent dresser after drug administration. Photographs were taken for each wound every other day from Day 0. Photos were scanned into a computer, and wound areas calculated using ImageJ image analysis software (National Institutes of Health, China).

The unhealed wound area was expressed as a percentage of the original wound area:

Aₜ/A₀×100%,

where A₀ and Aₜ refer to the initial area at Day 0 and the wound area at the date of measurement (time t), respectively.

Samples were taken at Day 4 and Day 7 post-wound infliction. The mice were sacrificed and the wound tissue taken by the same biopsy punch used to create the wound. Then 5 mm of tissue was cut off from the center of the sample and was preserved in 10% neutral buffered formalin (Nanchang Rain Dew Experimental Equipment Co., Ltd., Nanchang, Hubei Province, China), and analyzed by histological embedding (HE) in paraffin wax, sectioning and staining.

HE and Masson stained paraffin sections were analyzed under an optical microscope. Skin regeneration, fibroblastic proliferation, collagen regeneration scores and inflammation scores were estimated.

The rest of the samples were stored at −80° C. for further analysis. Tissue was cut into small pieces and liquid nitrogen was added to increase brittleness. 9 mL of normal saline was added to 1 g of tissue, which was then ground using a Tissuelyser (Shanghai Jingxin Industrial Development Co., Ltd., Shanghai, China) at 55 Hz for 60 seconds, followed by centrifuging at 8000 rpm for 10 minutes at 4° C.

The supernatant was collected, and the extracted protein was used for ELISA analysis using standard ELISA test kits and an ELISA reader (SH-1000 Hitachi, Japan). Vascular endothelial growth factor (VEGF), transforming growth factor beta 1 (TGF-β1) and hydroxyproline (Hyp) were assayed. The ELISA kits were purchased from Beijing 4A Biotech Co., Ltd. (Beijing, China).

The effects of Compound B on wound healing are show in Table 4, and in FIG. 2, which show the ratio of remaining wound area of initial wound in different groups (+SD in the case of Table 4).

TABLE 4

| Group | Model | rhEGF | Compound A | Compound B |
|---|---|---|---|---|
| Day 0 | 86.4 ± 5.4 | 82.0 ± 2.2 | 77.2 ± 4.1 | 79.4 ± 3.4 |
| Day 1 | 100 ± 7.2 | 100 ± 9.9 | 100 ± 17.1 | 100 ± 4.5 |
| Day 2 | 96.7 ± 3.6 | 94.1 ± 4.3 | 81.2 ± 11.2 | 96.7 ± 5.6 |
| Day 3 | 89.6 ± 3.1 | 78.6 ± 7.3 | 74.2 ± 0.9 | 76.7 ± 4.8 |
| Day 4 | 73.9 ± 3.8 | 61.5 ± 3.3 | 60.9 ± 7.3 | 66.5 ± 1.4 |
| Day 5 | 66.2 ± 5.9 | 53.0 ± 1.0 | 50.5 ± 11.3 | 55.8 ± 1.3 |
| Day 6 | 56.2 ± 5.3 | 47.2 ± 5.8 | 45.8 ± 4.7 | 51.9 ± 1.7 |

The above data show that a low dose of Compound B improves wound healing. The improvement rate was defined as (remaining wound ratio in treatment group/remaining wound ratio in model group)×100%. At Day 4, the improvement rate in the Compound B group was about 10%.

5 The wound Hyp (μg/mg) content, which is an indicator of collagen regeneration, is shown in Table 5 below and in FIG. 3.

TABLE 5

| | | | | | Compound | Compound |
|---|---|---|---|---|---|---|
| | | control | model | rhEGF | A | B |
| Mean | Day 4 | 4.070 | 1.551 | 1.808 | 3.676 | 2.039 |
| | Day 7 | 4.070 | 2.355 | 2.679 | 2.694 | 2.810 |
| SD | Day 4 | 0.980 | 1.100 | 0.790 | 0.951 | 0.965 |
| | Day 7 | 0.980 | 1.021 | 0.594 | 0.215 | 1.067 |

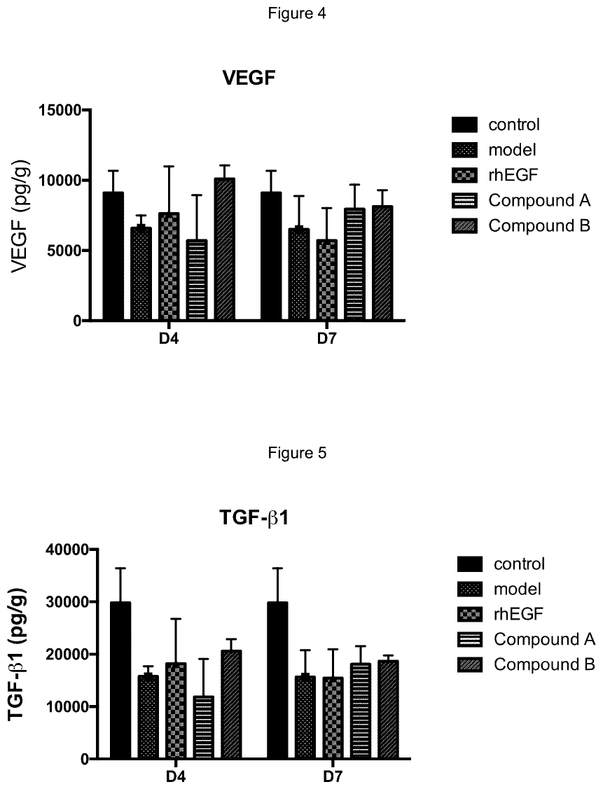

The result for VEGF content (μg/g) are tabulated in Table 6 below and are shown graphically in FIG. 4.

TABLE 6

| | | | | | Compound | Compound |
|---|---|---|---|---|---|---|
| | | control | Model | rhEGF | A | B |
| Mean | Day 4 | 9091.3 | 6598.0 | 7614.2 | 5709.7 | 10076.3 |
| | Day 7 | 9091.3 | 6500.1 | 5710.2 | 7949.5 | 8117.9 |
| SD | Day 4 | 1575.9 | 897.4 | 3362.9 | 3229.8 | 976.1 |
| | Day 7 | 1575.9 | 2375.4 | 2306.1 | 1734.8 | 1163.6 |

The result showed that Compound B has increased the VEGF production in the wounded tissue for about 56.7% in comparison with that in model group.

5 The result for TGF-β1 content (pg/g) are tabulated in Table 7 below and are shown graphically in FIG. 5.

TABLE 7

| | | | | | Compound | Compound |
|---|---|---|---|---|---|---|
| | | control | Model | rhEGF | A | B |
| Mean | Day 4 | 29832.2 | 15764.4 | 18190.6 | 11866.2 | 20571.8 |
| | Day 7 | 29832.2 | 15639.4 | 15443.1 | 18074.8 | 18659.6 |
| SD | Day 4 | 6554.9 | 1946.1 | 8564.5 | 7216.3 | 2310.6 |
| | Day 7 | 6554.9 | 5141.7 | 5482.1 | 3477.0 | 1123.0 |

### Example 4

### Liquid Spray to Treat Allenic Dermatitis.

Compound B (see Example 1 above) was dissolved in Water for Injection (WFI; prepared from a TC-RO-0.25/h-2 Water Treatment System, Yangzhou Tiancheng Water Treatment Devices & Engineering Co., Ltd., Yangzhou, China). The concentration was 0.3 mg/mL.

The enrolled subjects in the study had allergic skin. Prior to treatment, the patients presented symptoms including skin redness, itching and swelling. Subjects were required to use the liquid spray in the morning and the evening after facial cleansing.

The subjects all felt that itching was relieved within 8 minutes of the first use. Symptoms of capillary congestion disappeared and the patients' faces were no longer red and swollen after 1 day of use.

This experiments showed that a liquid spray comprising a compound of the invention relieves itching quickly and reduce redness, swelling and other symptoms caused by allergy.

### Example 5

### Cream to Treat Closed Comedones

Compound B (10 mg; see Example 1 above) was first dissolved in WFI (10 g).

A mixture was then made comprising sorbitan stearate (2 g), cetyl alcohol (4 g), octanoic/capric glyceride (6 g) and glyceryl monostearate (3 g) (all Sinopharm Chemical Reagent Co. Ltd.), which was stirred and heated to 85° C. to melt the mixture completely.

Glycerin (5 g; Sinopharm Chemical Reagent Co. Ltd.) and ammonium acryloyldimethyltaurate/VP copolymer (0.13 g; Clariant Chemical (Guangzhou) Co., Ltd., China) were mixed with 68.86 g of WFI. This mixture was stirred and heated to 85° C. to provide a homogeneous colloidal suspension.

The copolymer/water mixture was added to the sorbitan stearate-containing mixture, which was then quickly stirred using emulsification equipment for 5 minutes. The resultant emulsion was cooled to room temperature.

The Compound B solution was then added to the resultant mixture with stirring to obtain the finished cream. The concentration of Compound B in the final cream was 0.1 mg/g.

Subjects with closed comedones (blocked pores) and in an acute attacking period of acne were enrolled in the study. Symptoms included sebaceous glands blocked by keratinocytes, forming a slightly hard lump and a protruding white head.

The cream was evenly smeared in the morning and evening after facial cleansing. The lumps disappeared after 1 day of use, demonstrating that a cream comprising a compound of the invention can be used to treat closed comedones.

### Example 6

### Determination of Antioxidant Capacity of Compound of the Invention

The antioxidant capacity of Compound B was determined using a 2,2-diphenyl-1-(2,4,6-trinitrophenyl) hydrazyl (DPPH) assay.

Compounds A and B were obtained as described in Example 3 above. The peptide powders were stored at −20° C. and dissolved in saline at concentrations as indicated below. DPPH was purchased from Sigma-Aldrich China, Shanghai. Other reagents employed and identified below were purchased from Shanghai Aladdin Bio-Chem Technology Co. Ltd., China.

The DPPH method is one of the most commonly used methods in determination of antioxidant capacity. DPPH is a stable, nitrogen-centred free radical with a dark purple solution and a maximum absorption peak at 517 nm. When a free radical scavenger is present in the reaction system, it can be paired with a single electron of DPPH., and the absorption peak at 517 nm is gradually dissipated.

The degree of colour change bears a stoichiometric relationship to the number of paired electrons. Thus, antioxidant activity can be measured according to the change of absorbance. The greater the inhibition rate, the stronger the antioxidant capacity.

6 mg of DPPH was weighed and placed into a 100 mL volumetric flask. This was made up to volume with 80% ethanol to prepare a solution with a 0.06 mg/mL concentration. This was stored at 4° C. in the dark.

Peptide solutions were prepared by dissolving 0.6 g of peptide powder in 1 mL of distilled water.

200 μL of peptide solution was added to 3.8 mL of the DPPH solution. The solution was kept at room temperature for 1.5 hours in the dark. The absorbance at 517 nm was measured and was denoted as A1. The absorbance of the blank control sample was denoted as A0. Thus, the percentage of the DPPH remaining is calculated as: Clearance rate (%)=[A0−A1]/A0×100

The DPPH clearance rate for Compound A was 52.11%. That for Compound B was much less, at 8.9%, representing a significant improvement in antioxidant capacity.

### Example 7

### Stability of Compound of the Invention

Stabilities of Compounds A and B (obtained as described in Examples 1 and 3 above) were tested as follows.

10 mg of the compounds were each dissolved in 1 mL of distilled water to make a stock solution with a concentration of 10 mg/mL. Then, 200 μL of the stock solutions were diluted with 20 mM phosphate buffer (PB; Sigma-Aldrich China) with different pHs as indicated in Table 8 below.

TABLE 8

| | | | |
|---|---|---|---|
| No. | pH value | Buffer volume | Final pH tested |

Compound A

| | | | |
|---|---|---|---|
| 1 | 5.3 | 1800 μL | 5.3 |
| 2 | 7.2 | 1800 μL | 7.2 |
| 3 | 8.1 | 1800 μL | 8.1 |

Compound B

| | | | |
|---|---|---|---|
| 4 | 5.3 | 1800 μL | 5.3 |
| 5 | 7.2 | 1800 μL | 7.2 |
| 6 | 8.2 | 1800 μL | 8.1 |

All of the samples above were filtered through a 0.22 μm membrane (SLGV033RS; Sigma-Aldrich China) to remove potential bacteria in the solution on a super-clean bench after being prepared.

All the samples above were stored at room temperature and, three days later, were transferred to a 45° C. oven after initial sample analysis by HPLC (Column: Angilent ZORBAX Eclipse XDB-C18 (4.6×250 mm; 5 μm) (SN: USNH008244); buffer: A: 0.1% TFA in Water; B: 0.1% TFA in ACN; gradient: 0-25 minutes: 5%-30% B; 25-30 min: 100% B; flow rate: 1 mL/min; detection wavelength: 220 nm; sample volume: 20 μL).

The stability of the above samples was studied by observing the change of colour (Table 9), HPLC peak areas (Table 10) and purity (Table 11). The pH values for all samples were measured as being stable during the testing period.

TABLE 9

| | Compound A | | | Compound B | | |
|---|---|---|---|---|---|---|
| Day | No. 1 | No. 2 | No. 3 | No. 4 | No. 5 | No. 6 |
| 0 | NO | NO | NO | NO | NO | NO |
| 1 | NO | + | ++ | NO | NO | NO |
| 3 (Transfer | NO | + | ++ | NO | NO | NO |
| to 45° C.) | | | | | | |
| 6 (45° C.) | NO | ++ | ++ | NO | NO | NO |

NO means no colour changed was observed. + means a slight colour change is observed. ++ means a significant colour change is observed.

As shown in Table 9, the Compound A solutions became darker with time, particularly at higher pHs. The colour for rest of samples remained unchanged.

TABLE 10

| | Compound A | | | Compound B | | |
|---|---|---|---|---|---|---|
| Day | No. 1 | No. 2 | No. 3 | No. 4 | No. 5 | No. 6 |
| 0 | 100% | 100% | 100% | 100% | | 100% |
| 1 | 100.1% | 89.0% | 64.8% | 100.0% | | 98.9% |
| 3 (Transfer | 100.3% | 41.7% | 21.7% | 99.9% | 100% | 99.2% |
| to 45° C.) | | | | | | |
| 6 (45° C.) | 99.6% | 11.5% | 3.8% | 102.4% | 100.6% | 97.0% |

The peak area presented in Table 10 above as an area % of Day 0's result represents the peptide concentration. As shown in Table 10, peak areas of three samples of Compound B were almost unchanged. For Compound B, the peak area at pH 5 was unchanged, but at pH 7.2 and pH 8.1, the peak areas decreased significantly, which indicates that the concentration of peptide decreased with time.

TABLE 11

| | Compound A | | | Compound B | | |
|---|---|---|---|---|---|---|
| Day | No. 1 | No. 2 | No. 3 | No. 4 | No. 5 | No. 6 |
| 0 | 99.4% | 98.9% | 96.3% | 99.2% | 99.6% | 99.5% |
| 1 | 99.2% | 92.1% | 61.0% | 99.2% | 99.3% | 99.4% |
| 3 (Transfer | 99.4% | 94.8% | 52.5% | 98.4% | 98.7% | 98.4% |
| to 45° C.) | | | | | | |
| 6 (45° C.) | 98.0% | 13.8% | 3.9% | 99.0% | 98.2% | 96.4% |

When the peptides are oxidized, the HPLC peak shifts and the purity of peptide decreases. As shown in Table 11 above, the results show that most of Compound A was oxidized at pH 7.2 and pH 8.3.

It can be seem from the above results that the compound of the invention is stable at all pH values, but Compound A is stable only at pH 5.3.

### Example 8

### Compound B/Montelukast Dressing

An ointment was made by first dissolving montelukast sodium (200 mg; Arromax Pharmatech Co., Ltd, Suzhou, China) in polyethylene glycol 400 (20.0 g; Sinopharm Chemical Reagent Co. Ltd) with stirring. Compound B (16 mg; see Example 1 above) was then added to this solution.

Polyethylene glycol 3350 (21.3 g; Sinopharm Chemical Reagent Co. Ltd) was dissolved in in polyethylene glycol 400 (58.5 g) by heating to 60° C. with stirring. After this solution had cooled to 40-50° C., the solution containing Compound B and montelukast was added to it with stirring, followed by mixing for 5 to 10 minutes. Cooling to room temperature gave the finished product.

The ointment was coated evenly on a gauze with a flat plate. Cooling to room temperature gave the finished dressing.

### Example 9

### Compound B/Trypsin Spray

Compound B (30 mg; see Example 1 above) was dissolved in 10 mL of water. Trypsin (30 mg; Sichuan Deebio Pharmaceutical Co. Ltd., Guanhan, Sichuan, China) was dissolved in another 10 mL of water.

Calcium chloride (0.1 g), ethanol (0.5 g), water-soluble menthol (0.01 g), lactic acid 0.01 g and glycerol (30 g) (all Sinopharm Chemical Reagent Co. Ltd), were mixed together in 49.32 mL of water.

The solutions containing Compound B and trypsin were added to the mixture with stirring to provide a liquid for spraying.

### Example 10

### Compound B/Trpysin Aerosol for Inhalation

A aerosol formulation was prepared essentially as described in Example 9 above, from 20 mg of Compound B and 30 mg of trypsin, this time without glycerol and in a total of 99.32 mL of water.

### Example 11

### Common Cold Study

The subject that was enrolled in the study had a viral infection that was self-diagnosed as the common cold. The subject presented symptoms, including cough, sore throat and loss of voice for 2 days. Other anti-cold drugs had not been used prior to treatment.

The subject was required to use the liquid spray of Example 10 above in the morning and the evening.

On each use, 3 mL of the spray was poured into an atomizer storage tank which was attached to an atomizer. The atomizer was then opened and a mask was placed over the subject's mouth and nose.

The subject felt that throat pain was relieved after the first use. After the second use, the subject no longer felt pain in the throat and could speak, showing a liquid spray comprising a compound of the invention can relieve cough and sore throat symptoms caused by the common cold.

### Example 12

### Idiopathic Pulmonary Fibrosis (IPF) Model

Experimental animals and grouping: 40 adult male Sprague Dawley rats, after 7 days of adaptive feeding, were divided into 4 groups: sham-operation group (Sham), IPF model group (no treatment; Model), test group (Compound B) and positive control group (Positive Control).

The test were administered Compound B, at a dose of 130 μg/rat, which was introduced by aerosolized inhalation. Oral administration of pirfenidone (Etuary®, Beijing Continent Pharmaceutical Co., Ltd., Beijing, China) as a 120 mg/kg single-bolus dose served as the positive control.

A pulmonary fibrosis model was established by intratracheal instillation of bleomycin. The rats were anaesthetised and placed on an operating table in the supine position, to expose the trachea. Bleomycin (5 mg/kg) saline solution was injected into the trachea through the gap between the tracheal cartilage rings. The sham-operation group were given an equal volume of normal saline. Shortly after administration, the rats were lifted vertically and were rotated, in order to evenly disperse the drug. Once the rats had recovered, after approximately 5 days, they were administrated the different drugs according to the plan for 28 days, consecutively. The experimental plan was showed in Table 12.

TABLE 12

| Group | treatment | administration | dose | |
|---|---|---|---|---|
| Sham | Saline | Aerosolized inhalation | / | |
| Model | Saline | Aerosolized inhalation | / | |
| Positive Control | Pirfenidone | Gavage | 250 | mg/kg |
| Compound B | Compound B | Aerosolized inhalation | 130 | μg/Rat |

The exhaust volume of the atomizing device was 0.15 mL per minute. The inhalation time for each rat was 1 minute.

At Day 29 following drug administration, rats were anesthetized by i.p. injection of chloral hydrate. After orbital blood collection, the rats were sacrificed. The thoracic cavity was quickly opened and the lung was collected for further analysis. The lung wet weight was measured, and the lung coefficient was calculated (lung wet weight/rat weight×1000). The tissue was stored in refrigerator at −80° C. for further use.

A piece of lung tissue was accurately weighed, and 9 times the weight of saline was added. Then, the tissue was homogenized and centrifuged for 10 minutes at 3000 rpm. The homogenate was used to detect the expression of the transformation growth factor (TGF-β1), tumor necrosis factor-α (TNF-α) and alpha-smooth muscle actin (α-SMA). Standard ELISA methods were used for the detection. The contents of malonaldehyde (MDA) and the activity of superoxide dismutase (SOD) in lung tissue were also detected.

The effect of Compound B on TNF-α, α-SMA, TGF-β1 content in tissues of bleomycin-induced pulmonary fibrosis in rats are shown in Table 13.

TABLE 13

| | TNF-α (pg/mg | α-SMA | TGF-β1(ng/mg |
|---|---|---|---|
| | protein) | (pg/mg protein) | protein) |
| Sham | 181.85 ± 27.34 | 9.37 ± 0.72 | 0.41 ± 0.06 |
| Model | 301.69 ± 28.97 | 13.71 ± 1.41 | 0.55 ± 0.05 |
| Compound B | 187.91 ± 12.20 | 10.85 ± 1.42 | 0.40 ± 0.02 |
| Positive Control | 208.41 ± 17.73 | 10.98 ± 0.93 | 0.41 ± 0.03 |

The results show that Compound B inhibits the production of TNF-α, α-SMA, and TGF-β1 in lung tissue of bleomycin-induced pulmonary fibrosis in rats. The effects of inhibition were almost equivalent to that of the known IPF drug, pirfenidone.

The effect of Compound B on the lung coefficient, MDA, and SOD contents are shown in Table 14.

TABLE 14

| | lung coefficient | MDA (U/mg | SOD (U/mg |
|---|---|---|---|
| | (%) | protein) | protein) |
| Sham | 4.85 ± 0.26 | 0.12 ± 0.02 | 51.22 ± 1.07 |
| Model | 6.49 ± 0.69 | 0.66 ± 0.08 | 41.89 ± 2.82 |
| Compound B | 5.14 ± 0.37 | 0.17 ± 0.04 | 48.64 ± 1.85 |
| Positive Control | 5.54 ± 0.51 | 0.24 ± 0.05 | 48.59 ± 2.36 |

The results showed that the lung coefficient value was lower in the Compound B group than that in the Model group, which indicates that Compound B can reduce edema. Compared to the Model group, the lower MDA value and higher SOD value in the Compound B group show that Compound B has an antioxidant effect, by increasing SOD production and reducing lipid oxidation.

### Example 13

### Antitussive Experiment—Ammonia Induced Cough Method in Mice.

36 ICR mice were randomly divided into 3 groups according to their body weight, labelled a CMC-Na (Negative) control group, a dextromethorphan hydrobromide (Positive) control group and a Compound B (B) group. Each group contained 12 mice, with 6 males and 6 females in each.

Compound B was administered via atomization inhalation using a YLS-8A Multifunctional cough and sputum inducing device (Jinan Yiyan Science and Technology Co., Ltd.) (0.15 ml/min) for 1 minute, once daily for 5 days. And the positive control drug was administered once daily by intragastric administration at 10 mg/kg for 5 days.

The mice were placed in an inverted beaker 1, 2, and 4 hours after the last drug administration. 1 mL of aqueous ammonia (25.0 to 28.0%) was placed on top of a boiling water bath and was evaporated into the beaker.

The time for mice to receive ammonia exposure was determined by a method characterized by analyzing data immediately after one test or a batch of tests. The results of the previous analysis determined how to conduct the next test or batch of tests. In this way, the process of experiment-analysis-experiment proceeded sequentially:
(a) the time taken for the previous mouse to cough determined the time scheduled (which was shorter) for the next mouse to receive ammonia stimulation; or
(b) if the former mouse did not cough, the subsequent one would be stimulated for a longer time.

The difference between the logarithmic value of two adjacent time periods was 0.1.

The number of coughs within 1 minute was detected using a stethoscope. If a mouse coughed three or more times over one minute, this was labelled as called ‘cough’, and less than three times in one minute was labelled ‘cough free’.

The EDT₅₀ was defined as the ammonia stimulating time at which half mice developed a “cough”, calculated by the equation:

EDT₅₀=lg⁻¹c/n

(c equals the sum of r and x, r is the number of animals in each stimulating time group, x is the logarithm of stimulation time and n was the number of animals in each group). lg is a base 10 logarithmic value. The results shown in Table 15 below.

TABLE 15

| | | | Time after admin. | | | | | |
|---|---|---|---|---|---|---|---|---|
| | | | 1 hour | | 2 hours | | 4 hours | |
| | | | EDT₅₀ | R | EDT₅₀ | R | EDT₅₀ | R |
| | Dose | | (s) | (%) | (s) | (%) | (s) | (%) |
| Negative | | | 20.7 | | 21.5 | | 22.4 | |
| Positive | 10 | mg/kg | 50.1 | 241.7 | 44.7 | 207.3 | 41.4 | 184.8 |
| B | 195 | μg/mouse | 28.2 | 131.7 | 28.2 | 131.8 | 26.9 | 100 [???] |

In Table 15, R is the EDT₅₀ in treatment group divided by the EDT₅₀ in the Control group expressed as a percentage. An R value of >130% means that the compound has an antitussive effect and an R value of >150% indicates that it has obvious antitussive effect.

The results show that Compound B has a positive effect on cough relief at 1 and 2 hours.

### Example 14

### Liquid Spray to Treat Common Cold

Compound B was dissolved in Injection water (WFI; from a TC-RO-0.25 T/h-2 water treatment system, Yangzhou Tiancheng Water Treatment Devices & Engineering Co., Ltd., Yangzhou, China). The concentration was 0.5 mg/mL. 5 mL was loaded into a plastic water spray bottle.

The enrolled subject in the study was inflicted with a common cold for 2 days prior to treatment. Other cold treatments were not used before treatment. The subject, who presented a cough, a sore throat and a loss of voice, was required to use the liquid spray (3 to 5 presses each time, in the morning and evening.

The subject felt that her throat pain was relieved after the first use. After the second use, the subject felt that her throat was no longer painful and could speak.

This experiment showed that a liquid spray comprising a compound of the invention relieves cough and sore throat symptoms caused by the common cold.

### Example 15

### Aerosol Inhalation to Treat Fever.

The same formulation as described in Example 14 above was administered to a subject who enrolled in the study having had got a fever (a body temperature over 38.5° C.) for 1 day. Other anti-fever medications were not used prior to treatment.

The subject was required to use the aerosol inhalation device in the morning and in the evening by pouring 3 mL of the formulation into an atomizer storage tank connected to an atomizer and a mask, which was worn over the mouth and nose.

The subject felt that within two hours, his body temperature had dropped to normal. Fever symptoms did not recur after 3 uses.

This experiment shows that an aerosol inhalation comprising a compound of the invention relieves fever.

### Example 16

### Liquid Spray to Relieve Operational Pain During Laser Surgery.

The enrolled subject was required to use 1 mL of a liquid spray comprising the same formulation as that described in Example 14 above in a water spray bottle on half of her face before and after a lattice laser operation to remove of facial melanin.

The subject reported that she did not feel pain during the operation on half of her face to which the spray was applied. Similarly, she felt no pain after the operation. Conversely, pain was felt during the operation on the half of her face that was not sprayed. Furthermore, after the operation, the pain lasted about 2 hours.

This experiment shows that a liquid spray comprising a compound of the invention relieves operation pain during laser surgery.

### Example 17

### Combination Treatment of Ulcerative Colitis

A gel comprising Compound B (obtained as described above) and mefp-1 (USUN Bio Co., Jiangyin, China), both at concentrations of 1 mg/g, were prepared by combining the active ingredients with methyl cellulose (2.5%), propanediol (11%), glycerol (11%) and acetic acid (pH regulator; up to 0.5 g) to give a gel precursor with a pH of 5.5. All excipients were obtained from Sinopharm Chemical Reagent Co. Ltd. The gel was made up with water for injection. The gel was packed into a disposable anal-intestinal drug delivery system.

Before treatment, the subject defecated more than 20 times per day and exhibited severe bleeding from, and ulceration within, the colon. The gel formulation was given by through the anus. After having been given one dose on the first day, the patient defecated just three time on the second day. The subject then received two further doses on the second day. By the third day, bleeding was controlled.

After 14 days of administration at two doses per day, the symptoms of the patient's ulcerative colitis had been alleviated and there was no further bleeding.

1. The compound
Ala-Lys-Pro-Ser-Tyr-Hyp-Hyp-Thr-Tyr-Lys,
a regioisomer, a stereoisomer, or a pharmaceutically- or cosmetically-acceptable salt thereof, for use as a pharmaceutical.

2. A compound for use as claimed in claim 1, wherein the compound is:

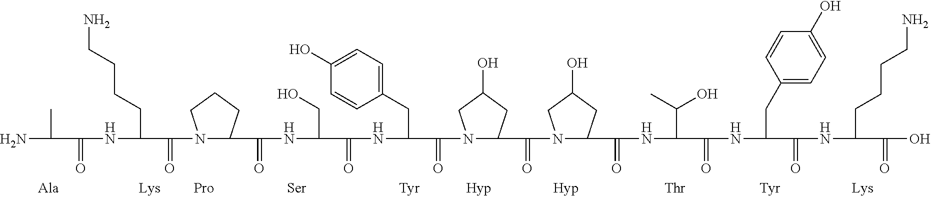 [SMILES: CC(N)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)cc1)C(=O)NC(CCCCN)C(=O)O)C(C)O]

3. A compound for use as claimed in claim 1 or claim 2, which is not in the form of a salt.

4. A pharmaceutical formulation comprising compound as defined in any one of claims 1 to 3, or a pharmaceutically- or cosmetically-acceptable salt thereof, and a pharmaceutically-acceptable adjuvant, diluent or carrier.

5. A pharmaceutical formulation including a compound as defined in any one of claims 1 to 3, or a pharmaceutically- or cosmetically-acceptable salt thereof; another antiinflammatory agent; and a pharmaceutically-acceptable adjuvant, diluent or carrier.

6. A kit of parts comprising components:
(A) a pharmaceutical formulation including a compound as defined in any one of claims 1 to 3, or a pharmaceutically- or cosmetically-acceptable salt thereof, in admixture with a pharmaceutically-acceptable adjuvant, diluent or carrier; and
(B) a pharmaceutical formulation including another antiinflammatory agent in admixture with a pharmaceutically-acceptable adjuvant, diluent or carrier, which components (A) and (B) are each provided in a form that is suitable for administration in conjunction with the other.

7. A compound as defined in any one of claims 1 to 3, or a pharmaceutically- or cosmetically-acceptable salt thereof, a formulation as claimed in claim 4 or claim 5, or a kit of parts as claimed in claim 6, for use in the treatment of inflammation, an inflammatory disorder, and/or of a disorder characterised by inflammation.

8. The use of a compound as defined in any one of claims 1 to 3, or a pharmaceutically- or cosmetically-acceptable salt thereof, a formulation as claimed in claim 4 or claim 5, or a kit of parts as claimed in claim 6, for the manufacture a medicament for the treatment of inflammation, an inflammatory disorder, and/or of a disorder characterised by inflammation.

9. A method of treatment of inflammation, an inflammatory disorder, and/or of a disorder characterised by inflammation, which method comprises the administration of a compound as defined in any one of claims 1 to 3, or a pharmaceutically- or cosmetically-acceptable salt thereof, a formulation as claimed in claim 4 or claim 5, or a kit of parts as claimed in claim 6, to a patient in need of such treatment.

10. A compound, formulation or kit of parts for use as claimed in claim 7, a use as claimed in claim 8, or a method as claimed in claim 9, wherein the inflammatory disorder is selected from psoriasis, acne, eczema, dermatitis, rhinitis, chronic obstructive pulmonary disease and ulcerative colitis.

11. A compound, formulation or kit of parts for use, a use or a method as claimed in claim 10, wherein the dermatitis is atopic dermatitis or steroid-dependent dermatitis.

12. A compound, formulation or kit of parts for use as claimed in claim 7, a use as claimed in claim 8, or a method as claimed in claim 9, wherein the disorder characterised by inflammation is a wound or a burn.

13. A compound, formulation or kit of parts for use, use or method as claimed in claim 12, wherein the wound is an abrasion, a scratch, an incision, a laceration, a skin puncture, an avulsion, a bruise, a scar or a blister, or itching associated with any of the foregoing.

14. A compound, formulation or kit of parts for use as claimed in claim 7, a use as claimed in claim 8, or a method as claimed in claim 9, wherein the condition or disorder characterised by inflammation is hemorrhoids.

15. A compound, formulation or kit of parts for use as claimed in claim 7, a use as claimed in claim 8, or a method as claimed in claim 9, wherein the disorder characterised by inflammation is a Viral infection.

16. A compound, formulation or kit of parts for use, use or method as claimed in claim 15, wherein the Viral infection is the common cold.

17. A compound as defined in any one of claims 1 to 3, or a pharmaceutically- or cosmetically-acceptable salt thereof, a formulation as claimed in claim 4 or claim 5, or a kit of parts as claimed in claim 6, for use in the treatment of idiopathic pulmonary fibrosis.

18. The use of a compound as defined in any one of claims 1 to 3, or a pharmaceutically- or cosmetically-acceptable salt thereof, a formulation as claimed in claim 4 or claim 5, or a kit of parts as claimed in claim 6, for the manufacture a medicament for the treatment of idiopathic pulmonary fibrosis.

19. A method of treatment of idiopathic pulmonary fibrosis, which method comprises the administration of a compound as defined in any one of claims 1 to 3, or a pharmaceutically- or cosmetically-acceptable salt thereof, a formulation as claimed in claim 4 or claim 5, or a kit of parts as claimed in claim 6, to a patient in need of such treatment.

20. A compound, formulation or kit of parts for use, use or method as claimed in any one of the preceding claims wherein the compound(s) or salt thereof, presented in a formulation or in a kit of parts is administered topically in the form of a topical formulation.

21. A compound, formulation or kit of parts for use, use or method as claimed in claim 20, wherein the adminstration is direct topical adminstration to a mucosal surface.

22. A compound, formulation or kit of parts for use, use or method as claimed in claim 21 (as dependent on any one of claims 1 to 10 or 17 to 19), wherein the adminstration is direct topical adminstration to the lung.

23. A compound, formulation or kit of parts for use, use or method as claimed in claim 20 (as dependent on any one of claims 1 to 16), wherein the adminstration is direct topical adminstration to the skin.

24. A formulation or kit of parts, use or method as claimed in any one of claims 20, 21 or 23 (as dependent on any one of claims 5 to 14), wherein the other antiinflammatory agent is montelukast of a pharmaceutically-acceptable salt thereof, and the condition is a wound, a burn or hemorrhoids.

25. A formulation or kit of parts, use or method as claimed in any one of claims 20 to 22 (as dependent on any one of claims 5 to 10, 13 or 17 to 19), wherein the other antiinflammatory agent is montelukast of a pharmaceutically-acceptable salt thereof, and the condition is chronic obstructive pulmonary disease or idiopathic pulmonary fibrosis. 26. A formulation or kit of parts, use or method as claimed in any one of claims 20 to 22 (as dependent on any one of claims 5 to 10), wherein the other antiinflammatory agent is mefp-1, and the condition is ulcerative colitis. 27. A formulation or kit of parts, use or method as claimed in claim 20 or claim 21 (as dependent on any one of claims 5 to 9, 15 or 16), wherein the other antiinflammatory agent is trypsin and the condition characterized by inflammation is a Viral infection. 28. A formulation or kit of parts, use or method as claimed in claim 27, wherein the Viral infection is the common cold. 29. A process for the preparation of a pharmaceutical formulation as defined in any one of claims 4 to 28, which process comprises bringing into association a compound as defined in any one claims 1 to 3 with the one or more adjuvant, diluent or carrier. 30. A process for the preparation of a kit of parts as defined in any one of claims 5 to 28, which process comprises bringing into association component (A) of the kit of parts with component (B) of the kit of parts.

31. A pharmaceutical formulation consisting essentially of:
an isolated decapeptide 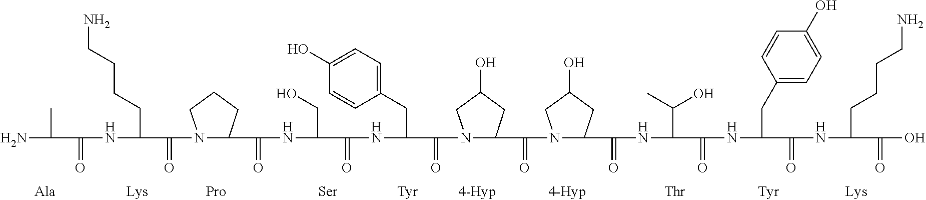 [SMILES: CC(N)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)cc1)C(=O)NC(CCCCN)C(=O)O)C(C)O]
(SEQ ID NO: 2), a stereoisomer, or a pharmaceutically acceptable salt thereof, optionally an anti-inflammatory agent that is different from the isolated decapeptide, and a pharmaceutically-acceptable adjuvant, diluent or carrier,
wherein the pharmaceutical formulation is suitable for topical application to skin or a mucosal surface.

32. The pharmaceutical formulation as claimed in claim 31, wherein said different anti-inflammatory agent is not present.

31. The pharmaceutical formulation of claim 31, wherein the formulation is in the form of a gel, lotion, paste, ointment, cream, foam, powder aerosol, or liquid aerosol.

34. A method of treatment of inflammation, an inflammatory disorder, and/or of a disorder characterised by inflammation, which method comprises the topical administration of the pharmaceutical formulation as claimed in claim 31 to a patient in need of such treatment.

35. The method as claimed in claim 34, wherein the inflammatory disorder is selected from psoriasis, acne, eczema, dermatitis, rhinitis, chronic obstructive pulmonary disease and ulcerative colitis.

36. The method as claimed in claim 35, wherein the dermatitis is atopic dermatitis or steroid-dependent dermatitis.

37. The method as claimed in claim 34, wherein the disorder is characterised by inflammation is a wound or a burn.

34. The method as claimed in claim 34, wherein the disorder characterised by inflammation is hemorrhoids.

39. The method as claimed in claim 34, wherein the formulation is administered topically to intact or disrupted skin.

40. A process for the preparation of the pharmaceutical formulation of claim 31, which process comprises bringing into association the isolated decapeptide with one or more pharmaceutically-acceptable adjuvant, diluent or carrier to form the pharmaceutical formulation suitable for topical application to skin or a mucosal surface.

41. A pharmaceutical formulation consisting of:
an isolated decapeptide 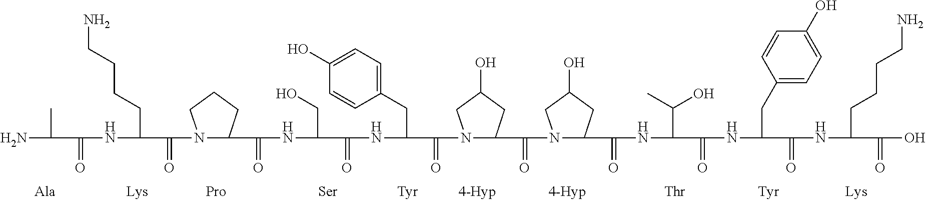 [SMILES: CC(N)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)cc1)C(=O)NC(CCCCN)C(=O)O)C(C)O]
(SEQ ID NO: 2), a stereoisomer, or a pharmaceutically-acceptable salt thereof, optionally an anti-inflammatory agent that is different from the isolated decapeptide, and a pharmaceutically-acceptable adjuvant, diluent or carrier,
wherein the pharmaceutical formulation is suitable for topical application to skin or a mucosal surface.

42. The formulation of claim 41, wherein the pharmaceutical formulation is in the form of a gel, lotion, paste, ointment, cream, foam, powder aerosol, or liquid aerosol.

43. A cosmetic formulation consisting essentially of:
an isolated decapeptide 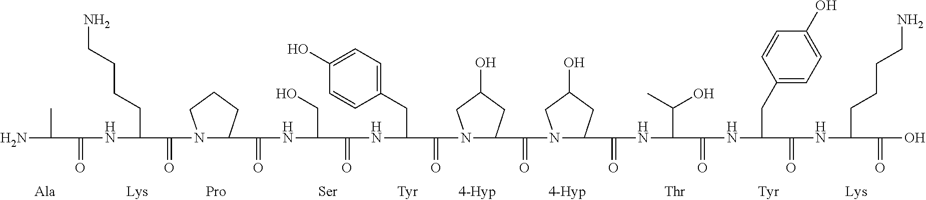 [SMILES: CC(N)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)cc1)C(=O)NC(CCCCN)C(=O)O)C(C)O]
(SEQ ID NO: 2), a stereoisomer, or a cosmetically-acceptable salt thereof, optionally an anti-inflammatory agent that is different from the isolated decapeptide, and a cosmetically-acceptable adjuvant, diluent or carrier,
wherein the cosmetic formulation is suitable for topical application to skin or a mucosal surface.

44. The cosmetic formulation as claimed in claim 43, wherein said different anti-inflammatory agent is not present.

45. The cosmetic formulation of claim 43, wherein the formulation is in the form of a gel, lotion, paste, ointment, cream, foam, powder aerosol, or liquid aerosol.

46. A method of treatment of inflammation, an inflammatory disorder, and/or of a disorder characterised by inflammation, which method comprises the topical administration of the cosmetic formulation as claimed in claim 43 to a patient in need of such treatment.

47. The method as claimed in claim 46, wherein the inflammatory disorder is selected from psoriasis, acne, eczema, dermatitis and rhinitis.

48. The method as claimed in claim 47, wherein the dermatitis is atopic dermatitis or steroid-dependent dermatitis.

49. The method as claimed in claim 46, wherein the disorder is characterised by inflammation is a wound or a burn.

50. The method as claimed in claim 46, wherein the formulation is administered topically to intact or disrupted skin.

51. A process for the preparation of the cosmetic formulation of claim 43, which process comprises bringing into association the isolated decapeptide with one or more cosmetically-acceptable adjuvant, diluent or carrier to form the cosmetic formulation suitable for topical application to skin.

52. A cosmetic formulation consisting of:
an isolated decapeptide 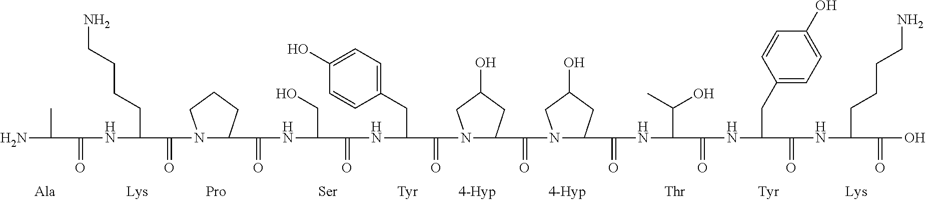 [SMILES: CC(N)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)cc1)C(=O)NC(CCCCN)C(=O)O)C(C)O]
(SEQ ID NO: 2), a stereoisomer, or a cosmetically-acceptable salt thereof, optionally an anti-inflammatory agent that is different from the isolated decapeptide, and
a cosmetically-acceptable adjuvant, diluent or carrier,
wherein the formulation is suitable for topical application to skin or a mucosal surface.

53. The cosmetic formulation of claim 52, wherein the formulation is in the form of a gel, lotion, paste, ointment, cream, foam, powder aerosol, or liquid aerosol.


SEQUENCE LISTING (2 sequences)

SEQ ID NO: 1 - protein, 10 aa, Mytilus edulis
  VAR_SEQ 6: note = Xaa at position 6 is 3-hydroxyproline
  VAR_SEQ 7: note = Xaa at position 7 is 4-hydroxyproline
  VAR_SEQ 9: note = Xaa at position 9 is dihydroxyphenylalanine
AKPSYXXTXK

SEQ ID NO: 2 - protein, 10 aa, Mytilus edulis
  VAR_SEQ 6..7: note = Xaa at positions 6-7 is 3-hydroxyproline or 4-hydroxyproline
AKPSYXXTYK